SEQUENCE LISTING (18 sequences)

SEQ ID NO: 1 - DNA, 18 bp, artificial sequence
  18 nucleotide primer hybridization site
ataccagcttattcaatt

SEQ ID NO: 2 - DNA, 18 bp, Artificial Sequence
  18 nucleotide primer hybridization site
agatagtaagtgcaatct

SEQ ID NO: 3 - DNA, 21 bp, artificial
  non-binding (control) probe
tacccctttaatcccaaaccc

SEQ ID NO: 4 - DNA, 41 bp, artificial sequence
  probe of the invention
atctaactgctgcgccgccgggaaaatactgtacggttaga

SEQ ID NO: 5 - DNA, 51 bp, artificial
  probe of the invention
acttattcaattcctgtgggaaggctatagaggggccagtctatgaataag

SEQ ID NO: 6 - DNA, 57 bp, artificial
  aptamer
cctgtgggaaggctatagaggggccagtctatgaataagatggcggactaatgtgta

SEQ ID NO: 7 - DNA, 52 bp, artificial
  aptamer
cctgtgggaaggctatagaggggccagtctatgaacaagatggttgatccgt

SEQ ID NO: 8 - DNA, 52 bp, artificial
  aptamer
agggggagcttgcgcgcatcaaggtgctaaacgaaagcctcatggcttctat

SEQ ID NO: 9 - DNA, 54 bp, artificial
  aptamer
cgagtgcggatgcaaacgccagacagggggacaggagataagaatagcgtgatg

SEQ ID NO: 10 - DNA, 35 bp, artificial
  aptamer
cgagtgcggatgcaaacgccagacagggggacagg

SEQ ID NO: 11 - DNA, 44 bp, artificial
  aptamer
accgacgacgaactatctatcactatcttacacatcatacctcg

SEQ ID NO: 12 - DNA, 51 bp, artificial
  aptamer
accgcagcgactatctcgactacattactagcttatactccgatcatctct

SEQ ID NO: 13 - DNA, 52 bp, artificial
  aptamer
agtcacacttagagttctaactgctgcgccgccgggaaaatactgtacggtt

SEQ ID NO: 14 - DNA, 52 bp, artificial
  aptamer
agtcacacttagagttctagctgctgcgccgccgggaaaatactgtacggat

SEQ ID NO: 15 - DNA, 52 bp, artificial
  aptamer
gagtgaagcaaggatgcaacctcggctccaacccgtgagagtcgcgaaactc

SEQ ID NO: 16 - DNA, 18 bp, artificial
  5′-primer
ataccagcttattcaatt

SEQ ID NO: 17 - DNA, 18 bp, artificial
  5′-primer
ataccagcttattcaatt

SEQ ID NO: 18 - DNA, 18 bp, artificial
  3′-primer
agattgcacttactatct

(19) United States
(12) Patent Application Publication
(10) Pub. No.: US 2011/0124015 A1
(43) Pub. Date: May 26, 2011

(54) APTAMER-BASED METHODS FOR IDENTIFYING CELLULAR BIOMARKERS

(76) Inventors: Weihong Tan, Gainesville, FL (US); Dihua Shangguan, Beijing (CN)

(21) Appl. No.: 12/948,382

(22) Filed: Nov. 17, 2010

Related U.S. Application Data
(63) Continuation of application No. 11/880,013, filed on Jul. 18, 2007, now abandoned.
(60) Provisional application No. 60/831,749, filed on Jul. 18, 2006.

(51) Int. Cl. G01N 33/574; C07K 14/00; G01N 33/50; G01N 30/02; H01J 49/26; G01N 27/26

(52) U.S. Cl. 435/7.23; 530/402; 436/86; 73/61.52; 250/282; 204/456

(57) ABSTRACT

In this invention, a biomarker discovery method has been developed using specific biotin-labeled oligonucleotide ligands and magnetic streptavidin beads. In one embodiment, the oligonucleotide ligands are firstly generated by whole-cell based SELEX technique. Such ligands can recognize target cells with high affinity and specificity and can distinguish cells that are closely related to target cells even in patient samples. The targets of these oligonucleotide ligands are significant biomarkers for certain cells. These important biomarkers can be captured by forming complexes with biotin-labeled oligonucleotide ligands and collecting the complexes using magnetic streptavidin beads, whereupon the captured biomarkers are analyzed to identify the biomarkers. Analysis of biomarkers include HPLC-Mass Spectroscopy analysis, polyacrylamide gel electrophoresis, flow cytometry, and the like. The identified biomarkers can be used for pathological diagnosis and therapeutic applications. Using the disclosed methods, highly specific biomarkers of any kinds of cells, in particular cancer cells, can easily be identified without prior knowledge of the existence of such biomarkers.

### CROSS-REFERENCE TO A RELATED APPLICATION

This application is a continuation application of co-pending U.S. application Ser. No. 11/880,013, filed Jul. 17, 2007; which claims the benefit of U.S. provisional application Ser. No. 60/831,749, filed on Jul. 18, 2006, both of which are hereby incorporated by reference in their entirety, including all figures, sequences and tables.

### GOVERNMENT SUPPORT

The subject matter of this application has been supported in part by U.S. Government Support under NIH GM66137 and NSF EF0304569. Accordingly, the U.S. Government has certain rights in this invention.

### BACKGROUND OF THE INVENTION

Molecular biomarkers are crucial for diagnosis of diseases and for predicting disease development. Potentially, some biomarkers can also be the targets of therapeutic agents for tumor-specific drug delivery in cancer treatments. Depending on the location, cancer markers can be present in the serum, such as the classic prostate-specific antigen (PSA) for prostate cancer (M. J. Barry, N. Engl. J. Med. 344, 1373 (2001)), or expressed directly on cells, such as the Her-2 receptor in breast cancer tumors (S. M. Pupa, E. Tagliabue, S. Menard, A. Anichini, J. Cell Physiol. 205, 10 (2005)). While the serum biomarkers have the advantage of simplifying cancer diagnosis in the form of blood tests, the cellular markers can be indispensable for cancer cell detection, whole-body tumor imaging, and targeted drug delivery.

Despite the enormous research efforts in the development of cancer biomarkers, a very limited number of molecules have been identified as effective markers for tumors. In fact, the only molecule approved by the US Food and Drug Administration (FDA) as tumor biomarker is PSA, which has been around for more than a decade (S. K. Chatterjee, B. R. Zetter, Future Oncol. 1, 37 (2005)). Previously, a biomarker would be considered ideal if it has high specificity, which means only the patients with the specific tumor will show the presence of the marker; and high sensitivity, which implies that most patients with such tumor should have this marker. However, it has been increasingly recognized that heterogeneity exists among individual patients, and even patients with the same type of cancer can have very different responses to cancer tests and therapies. This calls for personalized biomarkers to match the right patients for reliable cancer diagnosis and guided treatments.

Recent developments in biomarker discovery include genomic and proteomic approaches. The advances in DNA microarray technology have enabled mapping of gene expressions in tumor cells. By comparing to normal cells, differences in mRNA levels can be identified and linked to corresponding protein products, which form unique molecular signatures for the discovery of potential tumor markers. However, variants in the molecular signatures identified for similar biological systems have been observed among reports from different research groups (W. S. Dalton, S. H. Friend, Science 312,1165 (2006)). The main reasons behind this are likely insufficient number of samples and non-standardized technology platforms.

Similarly, proteomic approaches, often involving separation of serum proteins or whole-cell protein content, coupled with mass spectrometry based protein identification, can directly elucidate differences in protein expressions between tumor and normal samples (E. F. Petricoin et al., Lancet 359, 572 (2002), G. Zhou et al., Proteomics 5, 3814 (2005)). Though powerful in identifying biomarkers at the whole genome or proteome level, both approaches require the development of additional molecular probes for the markers before clinical diagnosis can be benefited practically and economically. In fact, these probes are essential in many applications of biomarkers such as whole-body imaging for specific tumors and targeted drug delivery. It is noteworthy that while these methods have been used for biomarker discovery for some time, the number of biomarkers identified and widely accepted is still very limited.

In relation to the need for simpler, expedient methods for the discovery and development of disease-specific biomarkers, a need exists for cancer-specific molecular probes. Specifically, multiple cancer-specific molecular probes are needed to report unique fingerprints of the tumor cells, especially given the complexity and diversity of cancer diseases, even those cancers in the same category (Alizadeh A. A. et al. Distinct types of diffuse large B-cell lymphoma identified by gene expression profiling. Nature 403, 503-511 (2000)). Identification and understanding of the molecular basis of diseased cells assist in providing reliable approaches toward effective diagnosis and treatments.

Even though many antibodies are available to recognize cellular markers, they were not intended for comprehensive recognition of molecular features of specific diseased cells, but rather individually developed at different times for various purposes. In fact, systematic production of a panel of antibodies for molecular differentiation of cancer cells is very difficult. For most diseases, there is a lack of reliable molecular probes that are specific enough to recognize subtle molecular differences among closely related diseases (Espina, V., Geho, D., Mehta, A. I., Petricoin, E. F. 3rd, Liotta, L. A., & Rosenblatt, K. P. (2005) Cancer Invest. 23, 36-46).

There are molecular-level differences between any two given types of cells such as normal vs. tumor cells. How to find out these differences and then use these differences to produce a full understanding of the molecular basis of diseases is critically important in molecular medicine. Despite the variety of clinical parameters used to classify human malignancies today, patients receiving similar diagnosis can have markedly different clinical courses and responses to treatments. This is believed to be due to subtle differences among patients that are not detectable using current technologies.

Behaviors of diseased cells are known to be originated from molecular compositions of the cells, and molecular heterogeneity within individual cancer diagnostic categories is already evident in complex genetic and proteomic alterations, thus differences at the molecular level should be used as a reliable way to differentiate cancer cells from normal cells, and even cancers in the same category. The profiling of these molecular characteristics should have a great potential in pinpointing cancer identity and providing personalized treatments. However, identification of molecular fingerprints of cancers remains extremely challenging, not to mention the development of corresponding molecular probes.

Cancers, as well as many other diseases, are originated from the mutations of human genes. As noted above, such genetic alterations result in not only different behaviors of the diseased cells, but also changes of the cells at the morphological and molecular levels. Traditionally, cancers are diagnosed mostly based on the morphology of tumor tissues or cells. However, these morphologic features are difficult to be used to carry out early cancer diagnosis, or to evaluate the complex molecular alterations that lead to cancer progression (Luo. J., Isaacs, W. B., Trent, J. M. & Duggan, D. J. (2003) Cancer Invest. 21, 937-949., Espina, V., Geho, D., Mehta, A. I., Petricoin, E. F. 3rd, Liotta, L. A., & Rosenblatt, K. P. (2005) Cancer Invest. 23, 36-46). Therefore, molecular characteristics, especially at the proteomic level, should be used to classify cancers because of the direct connection between genetic features and protein expression. Cancer diagnosis based on molecular features can be highly specific and extremely sensitive when incorporated with proper signal transduction and amplification mechanisms. Nonetheless, identification of molecular signatures of a particular cancer remains a great challenge if not impossible, which is reflected by the fact that very few biomarkers are available for effective cancer diagnosis.

Currently, diagnosis of leukemia is commonly based on morphologic evaluation supplemented by immunophenotype analysis (Belov, L., dela Vega, O., dos Remedios, C. G., Mulligan, S. P. & Christopherson, R. Immunophenotyping of leukemias using a cluster of differentiation antibody microarray. Cancer Res. 61, 4483-4489 (2001)). The expression of CD antigens on leukocytes is determined by flow cytometry with monoclonal antibodies. However, these antigens are usually expressed on both neoplastic cells and normal hematopoietic cells, and could not accurately reflect the molecular features of the cancer cells. In fact, no panels of monoclonal antibodies are available to reliably distinguish tumor cells from their normal counterparts. This is due to the technical difficulties in systematic development of antibodies for unknown surface biomarkers. To understand the molecular basis of cancers, novel approaches are needed to systematically generate new probes recognizing molecular signatures of cancers.

Molecular level differences are present between any two given types of cells, such as normal vs. tumor cells, tumor cell type 1 vs. type 2. These differences possess great significance in helping understand the biological processes and mechanisms of diseases. They could also be highly useful for disease diagnosis, prevention and therapy. However, identifying molecular differences between any two types of cells is not an easy task with current technologies. For example, discovery of unknown molecular features of diseased cells using molecular probes is almost impractical because, most of today's methodologies rely on known biomarkers for the development of corresponding molecular probes, which has been proved insufficient for addressing many emerging medical problems. Even if the molecular level differences can be identified, there is still a need to validate that the specific differences are indeed meaningful and vital for the desired biomedical property or the disease before any real clinical applications can be benefited.

Recently, aptamers have been suggested as being suitable for use in developing probes having high affinity and selectivity for target molecules. Aptamers are single-stranded DNA (ssDNA), RNA, or modified nucleic acids. They have the ability to bind specifically to their targets, which range from small organic molecules to proteins (Osborne, S. E. & Ellington, A. D. (1997) Chem. Rev. 97, 349-370, Nutiu, R. & Li, Y. (2005) Angew. Chem. Int. Ed. Engl. 44, 1061-1065, Wilson, D. S. & Szostak, J. W. (1999) Annu. Rev. Biochem. 68, 611-647). The basis for target recognition is the tertiary structures formed by the single-stranded oligonucleotides (Breaker, R. R. (2004) Nature 432, 838-845). Aptamers are obtained through an in vitro selection process known as SELEX (Systematic Evolution of Ligands by Exponential enrichment) (Ellington, A. D. & Szostak, J. W. (1990) Nature 346, 818-822, Tuerk, C. & Gold, L. (1990) Science 249, 505-510), in which aptamers are selected from a library of random sequences of synthetic DNA or RNA by repetitive binding of the oligonucleotides to target molecules. Aptamers have had many important applications in bioanalysis, biomedicine and biotechnology (Fang, X., Sen, A., Vicens, M. & Tan, W. (2003) ChemBioChem 4, 829-834, Guo, K., Wendel, H. P., Scheideler, L., Ziemer, G. & Scheule, A. M. (2005) J. Cell. Mol. Med. 9, 731-736, Yang, C. J., Jockusch, S., Vicens, M., Turro, N. & Tan, W. (2005) Proc. Natl. Acad Sci. USA 102, 102, 17278-83, Liu, J. W. & Lu, Y. (2006) Angew. Chem. Int. Ed. Engl 45, 90-94).

Selection of aptamers is through a process termed SELEX (systematic evolution of ligands by exponential enrichment) (A. D. Ellington, J. W. Szostak, Nature 355, 850 (1992)). Targets of aptamers are usually pure molecules such as proteins and small molecules. Recently, more complex biological species, such as red blood cells membrane and single protein on live trypanosomes, were also used as the targets in SELEX (B. J. Hicke et al., J. Biol. Chem. 276, 48644 (2001), D. A. Daniels, H. Chen, B. J. Hicke, K. M. Swiderek, L. Gold, Pro Natl Acad Sci. USA. 100, 15416 (2003), C. Wang et al., J. Biotechnol. 102, 15 (2003), K. N. Morris, K. B. Jensen, C. M. Julin, M. Weil, L. Gold, Proc. Natl. Acad. Sci. USA., 95, 2902 (1998), M. Homann, H. U. Goringer, Nucleic Acids Res., 27, 2006 (1999), M. Blank, T. Weinschenk, M. Priemer, H. Schluesener, J. Bio. Chem., 276,16464 (2001)).

Most aptamers reported so far have been selected using simple targets, such as a purified protein. Recently, aptamer-selection against complex targets, such as red blood cell membranes and endothelial cells, was also demonstrated (Morris, K. N. Jensen, K. B., Julin, C. M., Weil, M. & Gold, L. (1998) Proc. Natl. Acad. Sci. USA 95, 2902, Blank, M., Weinschenk, T., Priemer, M., & Schluesener, H. (2001) J. Biol. Chem. 276, 16464-16468, Daniels, D. A., Chen, H., Hicke, B. J., Swiderek, K. M. & Gold, L. (2003) Proc Natl Acad Sci USA. 100, 15416-21, Wang, C., Zhang, M., Yang, G., Zhang, D., Ding, H., Wang, H., Fan, M., Shen, B., & Shao, N. (2003) J. Biotechnol. 102, 15-22). Compared to molecular probes currently available for biomarker recognition, aptamers are emerging candidates with ample potential due to their high specificity, low molecular weight, easy and reproducible production, versatility in application, and easy discovery and manipulation (Jayasena, S. D. (1999) Clin. Chem. 45, 1628-1650). Currently the application of aptamers towards medical research and application is limited due to the lack of aptamers for systems of medical relevance.

### BRIEF SUMMARY

The subject invention provides methods for identifying cell biomarkers using highly specific and/or selective oligonucleotide ligands. The subject invention also discloses methods for producing such oligonucleotide ligands for use in identifying cell biomarkers. In certain related embodiments, the ligands are derived from an in vitro evolution process called SELEX (Systematic evolution of ligands by exponential enrichment) and can be synthesized in large scale by DNA synthesizer easily. Biomarkers of interest are subsequently captured in cell lysate by these oligonucleotide ligands, which are bound to a biotin-tag, and the resultant complexes are easily immobilized on a magnetic solid support using streptavidin coated magnetic beads.

According to the subject invention, the biomarkers discovered by this method are highly specific markers for certain cancer cells. They can be used as diagnosis reagents for all kinds of diseases, as targets for drug discovery, and as reagents for scientific research. Further, such biomarkers can be used in bioassays for diagnosis, therapeutically agents, the new discovery of diseases; etc.

In related embodiments, DNA aptamers are developed directly from tumor cells to function as highly specific and selective probes. These probes are tagged with biotin and subsequently bound to surface targets of leukemia cells to facilitate the discovery of potential new biomarkers via magnetic strategies. In certain embodiments, aptamers highly specific for a T-cell acute lymphoblastic leukemia (T-ALL) cell line are selected in accordance with the subject invention. Such T-ALL-aptamers have high affinity and excellent specificity. These molecular probes were utilized with magnetic strategies (biotin-labeling, streptavidin coated magnetic beads, and magnetic support stand) to capture and purify cancer cell membrane targets for the identification of disease biomarkers. Using the disclosed methods, a trans-membrane receptor protein tyrosine kinase 7 (PTK7) was identified to be the target of a T-ALL specific aptamer. The finding of high expression of PTK7 on T-ALL cells and the simultaneous development of an excellent aptamer probe for it assists in practical and reliable diagnosis of related leukemia.

In one embodiment, a novel strategy for preparing highly specific and selective molecular probes to a biomarker on a cell membrane is provided. In contrast to the conventional concept of biomarker/molecular probe discovery that requires prior knowledge regarding the target biomarker, the molecular probes of the subject invention are generated without any prior knowledge of possible biomarkers. The rationale behind this strategy is that molecular differences exist between any two given types of cells. Finding these differences generates personalized cell-specific molecular signatures for effective disease diagnosis and therapy as well as basic studies. Moreover, the resultant probes of the invention are highly useful for biomarker discovery as the specific binding sites for these molecules must be disease specific, generating the opportunity for biomarker elucidation.

In a related embodiment, the present invention provides a cell-based strategy (cell-SELEX) that generates a group of aptamers (designer DNA/RNA probes) that can specifically recognize an individual cancer cell type, without having prior knowledge about the cancer biomarker. These probes are then utilized to facilitate extraction, purification, and identification of the membrane biomarkers on the cancer cells. The identification of the membrane targets is realized through magnetized collection and separation and analysis via routine affinity chromatography coupled with mass spectrometry. Upon discovery of the cancer-specific biomarkers of the invention, aptamer probes (or antibodies) are generated for the recognition of these biomarkers for diagnosis of the corresponding disease, which greatly expedites the clinical application of the newly discovered biomarkers.

In the subject application, a group of new aptamers are provided for the recognition of minute molecular differences among leukemia patient samples. An easy and fast cell-based selection was used to evolve aptamers directly from leukemia cell lines. The selected aptamers have high affinity and specificity for the target cells, and can recognize closely related cells from real patient samples. Binding of the aptamers to the clinical samples formed distinct patterns. Leukemia patients pre-determined by antibodies to be in the same category were found to be different based on the aptamer-recognition profiles. Because of the direct connection between aptamer binding and cell surface target expression, the aptamers provided solid evidences of subtle molecular differences between closely related diseased cells. These subtle differences would be essential for not only accurate disease diagnosis, but also efficient personalized therapy.

The invention described below provides each of these advantages, among others, which will be apparent to those skilled in the art.

### BRIEF DESCRIPTION OF THE DRAWINGS

The file of this patent contains at least one drawing executed in color. Copies of this patent with color drawings(s) will be provided by the Patent and Trademark Office upon request and payment of the necessary fee.

FIG. 1A shows flow cytometry assays of CEM cells stained with anti-PTK7 PE and sgc8-FITC. FITC labeled Library and IgG-PE were used as negative control.

FIG. 1B shows flow cytometry assays of Ramos cells stained with anti-PTK7 PE and sgc8-FITC. FITC labeled Library and IgG-PE were used as negative control.

FIG. 2A shows flow cytometry assays of acute promyelocytic leukemia, NB-4 cells stained with anti-PTK7 PE and sgc8-FITC. FITC labeled Library and IgG-PE were used as negative control.

FIG. 2B shows flow cytometry assays of human acute lymphoblastic leukemia (T-cell line), Molt-4 cells stained with anti-PTK7 PE and sgc8-FITC. FITC labeled Library and IgG-PE were used as negative control.

FIG. 2C shows flow cytometry assays of human acute T cell leukemia, Jurkat cells stained with anti-PTK7 PE and sgc8-FITC. FITC labeled Library and IgG-PE were used as negative control.

FIG. 2D shows flow cytometry assays of human lymphoblastic leukemia (T-cell line), Sup-T1 cells stained with anti-PTK7 PE and sgc8-FITC. FITC labeled Library and IgG-PE were used as negative control.

FIG. 3A shows flow cytometry assays of Ramos cells nucleofected with plasma containing eDNA of PTK7, (left) without adding sgc8; (right) with added sgc8; the elliptical area highlights the cells who express PTK7.

FIG. 3B show flow cytometry assays of negative controls, (left) without plasma with nucleofection program; (right) with plasma without nucleofection program.

FIG. 4 shows Colloidal Blue-stained SDS-PAGE used to analyze the aptamer-assisted target purification. Lane 1, molecular markers; Lane 2, membrane extracts; Lane 3, protein capturing with the non-binding sequence; Lane 4, magnetic beads; Lane 5, protein capturing with sgc3b; and Lane 6, protein capturing with sgc8c.

FIG. 5 are graphical illustrations of the lack of competition between anti-PTK7 and sgc8c. Left: CEM cells binding with anti-PTK7-PE; Right: CEM cells binding with anti-PTK7-PE after incubation with unlabeled sgc8.

FIG. 6 is a schematic diagram of cell-based aptamer selection. Briefly, starting ssDNA pool (a DNA library containing a central randomized sequence of 52 nucleotides (nt) flanked by 18-nt primer hybridization sites: 5′-ATA CCA GCT TAT TCA ATT (SEQ ID NO:1)-(central randomized 52mer)-AGA TAG TAA GTG CAA TCT-3′) (SEQ ID NO:2) is incubated with target cells. After washing, the bound DNAs are eluted by heating in binding buffer. The eluted DNAs are then incubated with control cells for counter-selection. After centrifugation, the unbounded DNAs in supernatant are collected, and then amplified by PCR. The amplified DNAs are used for next round selection. The selection process is monitored using fluorescent imaging or flow cytometry. Once a significant enrichment is achieved after 10-20 rounds of selection, the pool will be cloned and sequenced. The sequences will then be tested with both target and control cells for the selection of useful aptamers. The aptamers will be further optimized and labeled with dye molecules before real applications.

FIG. 7 is a schematic representation of cell-based aptamer selection in accordance with the present invention. Briefly, ssDNA pool was incubated with CCRF-CEM cells (target cells). After washing, the bound DNAs were eluted by heating to 95°. The eluted DNAs were then incubated with Ramos cells (negative cells) for counter-selection. After centrifugation, the supernatant was collected and the selected DNA was amplified by PCR. The PCR products were separated into ssDNA for next round selection or cloned and sequenced for aptamer identification in the last round selection.

FIG. 8A shows flow cytometry assays used to monitor the binding of selected pool with CCRF-CEM cells (target cells) and Ramos cells (negative cells). The “unselected Lib”/green curve represents the background binding of unselected DNA library. For CEM cells, there was an increase in binding capacity of the pool as the selection was progressing, while there was little change for the control Ramos cells.

FIG. 8B shows confocal images of cells stained by the 20th round selected pool labeled with tetramethylrhodamine dye molecules. Top left: fluorescence image of CCRF-CEM cells; top right: optical image of CCRF-CEM cells. Bottom left: fluorescence image of Ramos cells; bottom right: optical image of Ramos cells.

FIG. 9A shows flow cytometry assays for the binding of the FITC-labeled sequence sga16 and sgc8 with CCRF-CEM cells (target cells) and Ramos cells (negative cells). The “Lib”/green curve represents the background binding of unselected DNA library. The concentration of the aptamers in the binding buffer was 250 nM.

FIG. 9B is a graphical illustration that uses flow cytometry to determine the binding affinity of the FITC-labeled aptamer sequence sga16 to CCRF-CEM cells. The nonspecific binding was measured by using FITC-labeled unselected library DNA.

FIG. 10A is a graphical illustration of flow cytometry assays for the binding of the FITC-labeled sequence sgc3 with CCRF-CEM cells (target cells) and Ramos cells (negative cells). The “Lib”/green curve represents the background binding of unselected DNA library. The second peak of the “sgc3” curve represents the sgc3 labeled subset of cells. The concentration of the aptamer in the binding buffer was 250 nM.

FIG. 10B shows fluorescence confocal images of CEM and Ramos cells stained by sgc3 labeled with TAMRA: (Left) fluorescence images and (Right) optical images for CCRF-CEM cells and Ramos cells respectively.

FIG. 10C shows flow cytometry assays for the binding of CCRF-CEM cells to aptamer sgc3 and monoclonal antibodies against CD5, CD7 and CD3. The yellow area represents the sgc3 labeled subset of cells. Aptamer sgc3 selectively bound to a subpopulation of CCRF-CEM cells, which expressed bright CD7 and CD5 but not CD3. The final concentration of sgc3 in the binding buffer was 250 nM.

FIG. 11 shows flow cytometry assays of the molecular recognition of CCRF-CEM cells and human bone marrow cells when incubated with the ETC-labeled sgc8 aptamer, sgc3 aptamer, and PerCP-labeled anti-CD45 antibody. The FITC-labeled aptamer sgc8, aptamer sgc3, and monoclonal antibodies were incubated with the target CCRF-CEM cells and/or bone marrow cells. The sgc8 (B) and sgc3(C) were able to recognize the target leukemia cells selectively when CCRF-CEM leukemia cells were mixed with cells from human bone marrow aspirates.

FIG. 12 shows flow cytometry assays of the molecular recognition of T-ALL cells in patient bone marrow aspirates with FITC-labeled sgc8 aptamer, sgc3 aptamer, sgc4 aptamer, sgd2 aptamer, sgd3 aptamer, and PE-labeled anti-CD7 antibody. The background was measured by using FITC-labeled unselected library. The red dots represent T-ALL cells.

FIG. 13A is a graphical illustration of the binding of aptamers sgc8 and sgc3 to trypsin treated CCRF-CEM cells. The concentration of the aptamers in the binding buffer was 250 nM.

FIG. 13B is a graphical illustration of the binding of aptamers sgc8 and sgc3 to proteinase K treated CCRF-CEM cells. The concentration of the aptamers in the binding buffer was 250 nM.

### BRIEF DESCRIPTION OF THE SEQUENCE LISTING

SEQ ID NO.1 is a single stranded DNA 18-nucleotides (nt) primer hybridization site that is combined with a central randomized sequence of 52 nucleotides for obtaining a probe of the invention.

SEQ ID NO:2 is a single stranded DNA 18-nucleotides (nt) primer hybridization site that is combined with a central randomized sequence of 52 nucleotides for obtaining a probe of the invention.

SEQ ID NO:3 is the nucleotide sequence of a non-binding (control) probe of the invention.

SEQ ID NO:4 is the nucleotide sequence of a probe of the invention.

SEQ ID NO:5 is the nucleotide sequence of yet another probe of the invention bound to biotin and two 18-atom-ethylene glycol spacers.

SEQ ID NOS:6-15 are nucleotide sequences of probes obtained using the methods of the invention.

SEQ ID NO:16 is the nucleotide sequence of a labeled 5′-primer.

SEQ ID NO:17 is the nucleotide sequence of a labeled 5′-primer

SEQ ID NO:18 is the nucleotide sequence of a 3′-primer.

### DETAILED DISCLOSURE

The subject invention provides methods for biomarker discovery. The methods of the invention use highly specific and/or selective biotin-labeled oligonucleotide ligands in combination with magnetic strategies for isolating and identifying biomarkers of interest. Specific magnetic strategies include the use of streptavidin coated magnetic beads that would bind to the biotin-labeled oligonucleotide ligands and magnetic support for collecting the magnetically-bound comlexes.

In one embodiment, the oligonucleotide ligands are firstly generated by whole-cell based SELEX technique. Such ligands can recognize target cells with high affinity and specificity and can distinguish cells that are closely related to target cells even in patient samples. The targets of these oligonucleotide ligands are significant biomarkers for certain cells. These important biomarkers are captured by forming complexes with biotin-labeled oligonucleotide ligands and collecting the complexes using magnetic streptavidin beads, whereupon the captured biomarkers are analyzed to identify the biomarkers.

Analysis of biomarkers include HPLC-Mass Spectroscopy analysis, polyacrylamide gel electrophoresis, flow cytometry, and the like. The identified biomarkers can be used for pathological diagnosis and therapeutic applications. Using the disclosed methods, highly specific biomarkers of any kinds of cells, in particular cancer cells, can easily be identified without prior knowledge of the existence of such biomarkers.

### Biomarker Identification and Development

Using cell-based aptamer selection (Cell-SELEX), a novel strategy for identifying biomarkers is presented which utilizes the differences at the molecular level between any two types of cells for the identification of molecular signatures on the surface of targeted cells. In certain specific embodiments, a group of aptamers has been generated for the specific recognition of leukemia cells. The selected aptamers can bind to target cells with an equilibrium dissociation constant (Kd) in the nM to pM range. The cell-based selection process is simple, fast, straightforward and reproducible, and, most importantly, can be done without prior knowledge of target molecules. The selected aptamers can specifically recognize target leukemia cells mixed with normal human bone marrow aspirates, and can also identify cancer cells closely related to the target cell line in real clinical specimens. The cell-based aptamer selection holds a great promise in developing specific molecular probes for cancer diagnosis and cancer biomarker discovery.

To find cancer-specific cell membrane biomarkers, a modified SELEX method was used to generate aptamers against whole leukemia cells. Briefly, a T-cell acute lymphoblastic leukemia (T-ALL) cell line CCRF-CEM was incubated with a DNA library containing around 10¹⁵ single-stranded random DNA sequences (D. Shangguan et al., Proc. Natl. Acari Sci. USA 103, (2006)). Sequences bound to the target cells were collected while the unbound sequences were washed away. The collected sequences then went through a counter-selection process where they were incubated with a control cell line (Ramos, a B-cell lymphoma cell line). Following that, the unbound DNAs were eluted and PCR amplified to form a new DNA pool. The counter-selection step is designed to make sure only the sequences that bind to the target cells but not the control cells will be enriched. The new DNA pool, with a better affinity for the target cells than the initial library, was incubated with the target cells again to start a new cycle of selection and counter-selection. This process was repeated for up to 20 cycles until a DNA pool with high affinity and good selectivity for the target cells was obtained.

Progress of the enrichment of the aptamer candidates was monitored using both flow cytometry and confocal microscopy. The final DNA pool was sequenced and a panel of aptamer sequences were determined. One of the aptamers with the highest affinity and specificity for the CCRF-CEM leukemia cells was named sgc8. Sgc8 was tested on normal bone marrow cell and a variety of Hematopoietic cancer cells including patient samples. Most of the leukemia cells closely related to the CCRF-CEM cells showed significant sgc8 binding, while almost all of the lymphoma cells and cells in normal bone marrow had negligible fluorescence from dye-labeled sgc8 (data not shown). This level of selectivity clearly indicates the presence of a highly specific molecular marker on the membrane of these leukemia cells.

The following example illustrates a procedure for practicing the invention. This example should not be construed as limiting the scope of the invention in any way. All percentages are by weight and all solvent mixture proportions are by volume unless otherwise noted.

### EXAMPLE 1

### Materials

### Cell Lines and Reagents

CCRF-CEM (CCL-119, T-cell lines, human acute lymphoblastic leukemia), Ramos, (CRL-1596, B-cell line, human Burkitt's lymphoma), Toledo (CRL-2631, B-cell line, human diffuse large cell lymphoma), Sup-T1(CRL-1942, T-cell lines, human lymphoblastic leukemia), Jurkat (TIB-152, human acute T cell leukemia), Molt-4 (CRL-1582, T-cell lines, human acute lymphoblastic leukemia), were obtained from ATCC (American Type Culture Collection). NB-4 (acute promyelocytic leukemia) was obtained from the Department of Pathology, University of Florida). All the cells were cultured in RPMI 1640 medium (ATCC) supplemented with 10% fetal bovine serum (FBS) (heat inactivated, GIBCO) and 100 IU/mL penicillin-Streptomycin (Cellgro). Cells were washed before and after incubation with wash buffer (4.5 g/L glucose and 5 mM MgCl₂ in Dulbecco's phosphate buffered saline with calcium chloride and magnesium chloride (Sigma)). Binding buffer used for selection was prepared by adding yeast tRNA (0.1 mg/mL) (Sigma) and BSA (1 mg/mL) (Fisher) into wash buffer to reduce background binding. Monoclonal anti-PTK7 antibody conjugated to R-phycocrythrin (PE) was purchased from Miltenyi Biotec Inc (Auburn, Calif., USA). Magnetic streptavidin beads were purchased from Dynal biotech ASA (Oslo, Norway). Homo sapiens PTK7 transcript variant PTK7-1 transfection-ready DNA was purchased from OriGene Technologies, Inc (Rockville, Md., USA). Cell Line Nucleofector Kit V was purchased from Amaxa Inc (Gaithersburg, Md., USA).

### Oligodeoxynucleotide Probe Synthesis

Biotin-Rb1 (B-TACCCCTTTAATCCCAAACCC (SEQ ID NO:3), B denotes biotin), a non binding sequence, Biotin-sgc8c (Biotin-S-S-S-S-ATCTAACTGCTGCGCCGCCGGGAAAATACTGTACGGTTAGA (SEQ ID NO:4), S denotes an 18-atom ethylene glycol spacer), and Biotin-sgc3b (Biotin-S-S-ACTTATTCAATTCCTGTGGGAAGGCTATAGAGGGGCCAGTCTATGAATAAG) (SEQ ID NO:5) were synthesized according to the standard phosphoramidite chemistry on an ABI 3400 synthesizer. Oligonucleotide building blocks were obtained from Glen Research Corporation (Virginia, USA). Purification was performed twice for each oligonucleotide using RP-HPLC with the DMT-on mode on a Varian HPLC system.

### Biomarker Purification and Identification

4×10⁸ CCRF-CEM cells were washed three times at 4° C. with PBS buffer, and lysed in 5 mL of hypotonic buffer [50 mM Tris-HCl (pH 7.5) containing protease inhibitors (0.1 mM PMSF, and 2 μg/ml pepstatin, leupeptin, and aprotinin)] at 4° C. for 30 min. After centrifugation, the debris were washed with 5 ml of hypotonic buffer for 3 times and dissolved in 1.5 mL of lysis buffer (PBS containing 5 mM MgCl₂ and 1% Triton X-100) at 4° C. for 30 min. After centrifugation, the supernatant was incubated with 150 pmole non-binding sequences Biotin-Rb1 in the presence of 1000-fold excess of 88mer random DNA sequence library (150 nmol) as a nonspecific competitor at 4° C. for 30 min, the protein-DNA complex was captured by incubating with 2 mg (200 μl) magnetic streptavidin beads at 4° C. for 15 min, and collected on a magnet stand. The resulting supernatant was incubated with 150 pmole Biotin-sgc8c at 4° C. for 30 min, and the protein-sgc8c complex was captured by incubating with 2 mg (200 μl) magnetic streptavidin beads at 4° C. for 15 min and collected on a magnet stand. The resulting supernatant was incubated with 150 pmole Biotin-sgc3b at 4° C. for 30 min, the protein-sgc3b complex was captured by incubating with 2 mg (200 μl) magnetic streptavidin beads 4° C. for 15 min, and collected on a magnet stand. The collected magnetic beads were washed with 1 ml PBS containing 5 mM MgCl₂ for 4 times. The proteins was eluted by heating in 30 μl loading buffer and analyzed by polyacrylamide gel electrophoresis (SDS-PAGE) stained with Colloidal Blue.

The aptamer-purified protein bands were excised and digested in situ, and analyzed by QSTAR LC-MS/MS and MASCOT® database search at the Protein Chemistry Core Facility, University of Florida.

### Flow Cytometric Analyses

5×10⁵ cells were incubated with excess anti-PTK7-PE and/or 200 nM FITC-labeled aptamer sgc8 in 200 μL of binding buffer (PBS containing 5 mM MgCl₂, 4.5 g/L glucose, 0.1 mg/mL yeast tRNA, 1 mg/mL BSA and 20% FBS) on ice for 30 min. Cells were washed twice with 0.7 ml of binding buffer (with 0.1% NaN₂), and suspended in 0.3 ml of binding buffer (with 0.1% NaN₂). The fluorescence was determined with a FACScan cytometer (Becton Dickinson Immunocytometry Systems, San Jose, Calif.) and 30000 events were measured for each cell sample. The FITC-labeled un-enriched ssDNA library and PE-labeled IgG were used as negative controls.

For competition experiments, 5×10⁵ cells were incubated with 2 μM unlabeled sgc8 in binding buffer on ice for 20 min, then anti-PTK7-PE was added and incubated for 30 min. After wash, the fluorescence of anti-PTK7-PE was determined with a FACScan cytometer and compared with that without the addition of sgc8.

### PTK7 Gene Transfection

2-4 μg of plasmid DNA containing full length cDNA of PTK7 (Vector: pCMV6-XL4) was transfected into 2×10⁶ Ramos and Toledo cells following the protocols of Cell Line Nucleofector Kit V. Positive control with vector pmaxGFP and 2 negative controls were used to assess the transfection efficiency. The transfected cells were analyzed by flow cytometry after 20 hour.

The target of sgc8 was previously proven to be a membrane protein since the CEM cells completely lost binding to sgc8 after being treated with trypsin or proteinase K (D. Shangguan et al., Proc. Natl. Acad. Sci. USA 103, (2006)), although the identity of the biomarker was still unknown. Following that, efforts were carried out to identify the target of sgc8 as provided in Example 1 above.

As described in detail above, CEM cells were lysed and the membrane content was dissolved in PBS buffer containing surfactant. An optimized and truncated DNA sequence of sgc8, sgc8c, which had identical binding properties as sgc8, was previously labeled with a biotin tag at the 5′-end and incubated with the solubilized membrane proteins. The binding complex of sgc8 and its target was then extracted using streptavidin coated magnetic beads. After wash, the streptavidin beads, along with the captured proteins, were heated in the loading buffer of SDS-PAGE and the eluted proteins underwent subsequent separation by SDS-PAGE (FIG. 4).

By comparing to control experiments, characteristic protein bands on the gel captured by sgc8 were digested and supplied to LC-MS/MS QSTAR analysis. MASCOT® database search was used to assign possible protein candidates to the MS results. Among the list of protein hits, PTK7-5 (an isoform of protein tyrosine kinase 7) received most attention because of several reasons.

First, it is a transmembrane receptor. Second, the size of the PTK7 (118 kD) correlates very well with the molecular weight obtained from the protein band on the SDS-PAGE gel. Also known as colon carcinoma kinase-4 (CCK-4), PTK7 was reported to have an increased expression level in metastatic colon carcinoma (K. Mossie et al., Oncogene 11, 2179 (1995)). It was also found to play a role in regulating planar cell polarity in vertebrates (X. Lu et al., Nature 430, 93 (2004)). PTK7 is believed to be important in the signaling mechanism during cancer development and metastasis (J. W. Jung, W. S. Shin, J. Song, S. T. Lee, Gene 328, 75 (2004)). However, its exact function is not clear, and there are no reports that have linked PTK7 over-expression to T-ALL cells.

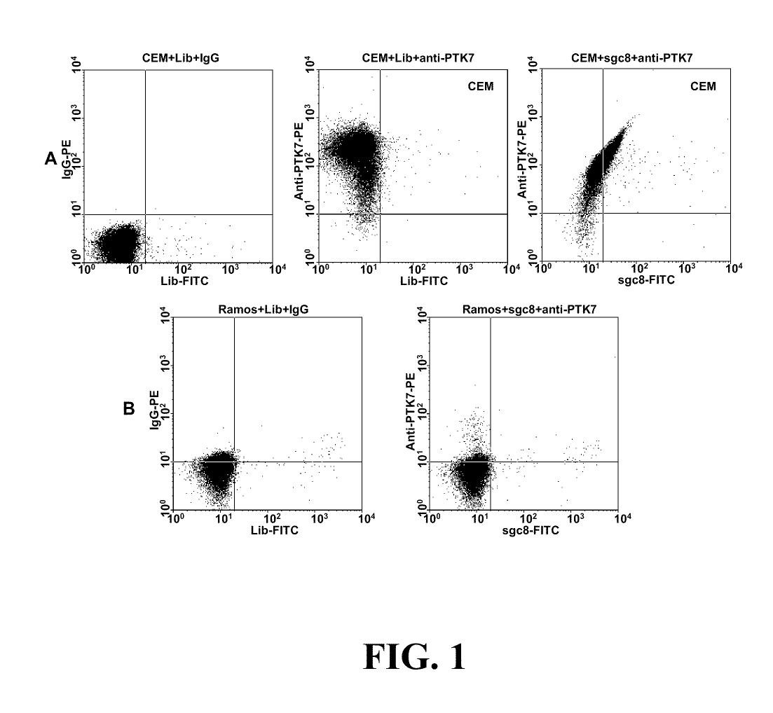

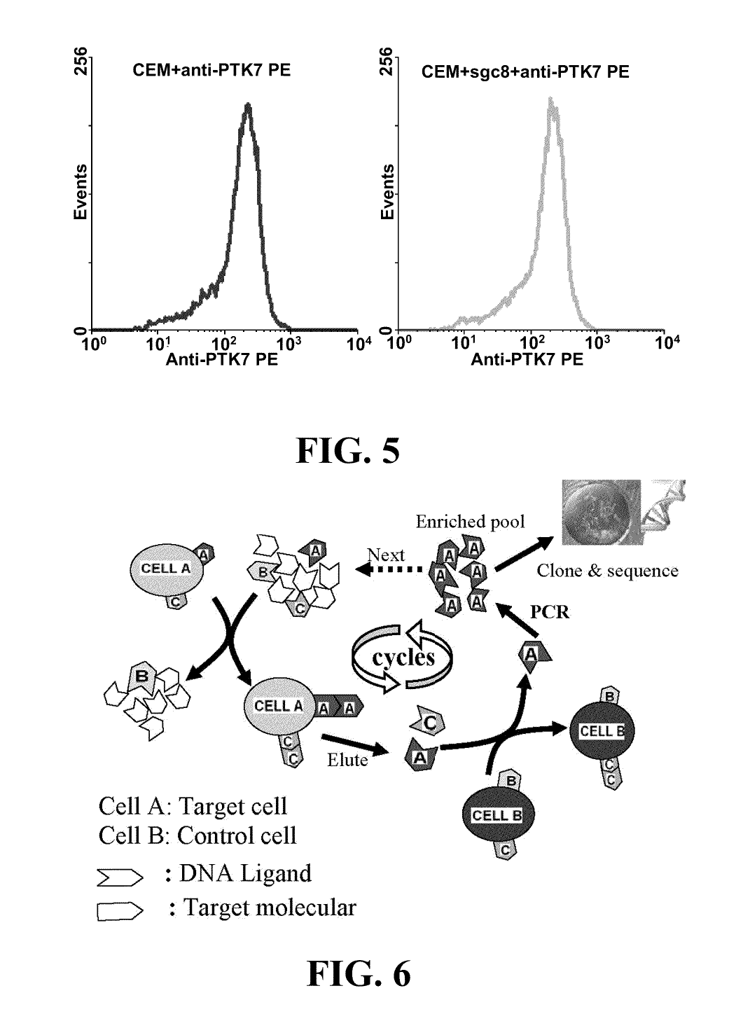

The presence of PTK7 on the CEM cells was first confirmed using the anti-PTK7 antibody labeled with a phycoerythrin (PE) dye (FIG. 1). To evaluate the interaction between sgc8 and PTK7, excess unlabeled sgc8 was used to compete with anti-PTK7-PE for CEM cell binding. Interestingly, flow cytometry results showed no obvious change in anti-PTK7-PE binding (FIG. 5), indicating there was no competition between sgc8 and anti-PTK7. To further investigate the possibility of co-binding of sgc8 and the antibody on PTK7, the aptamer was labeled with a FITC fluorophore and incubated with CEM cells along with anti-PTK7-PE. A flow cytometry analysis of the cells was then conducted and the resultant dot plot is shown in FIG. 1. Fluorescence signals from the FITC and PE channels displayed a linear relationship. An immediate fact drawn from the linearity is that cells with higher PTK7 expression also have better binding to sgc8. A possible explanation would be that sgc8-FITC and anti-PTK7-PE can bind to two different sites of the extracellular domain of PTK7 simultaneously, or sgc8-FITC can bind to a molecule that is tightly associated with. PTK7.

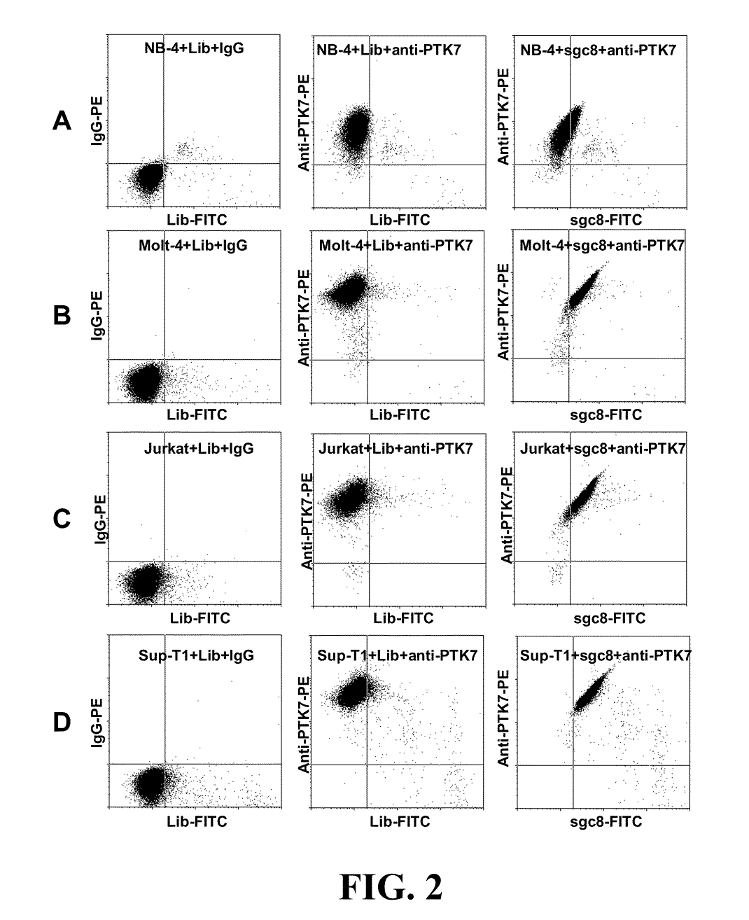

Control experiments were performed by exchanging anti-PTK7-PE or sgc8-FITC with a different antibody or aptamer, or using a different cell line. In any of these cases, the linearity was not seen (FIG. 1). On the other hand, a few cell lines previously tested to have sgc8 binding gave a very similar linear relationship between FITC and PE signals from sgc8 and anti-PTK7 respectively (FIG. 2).

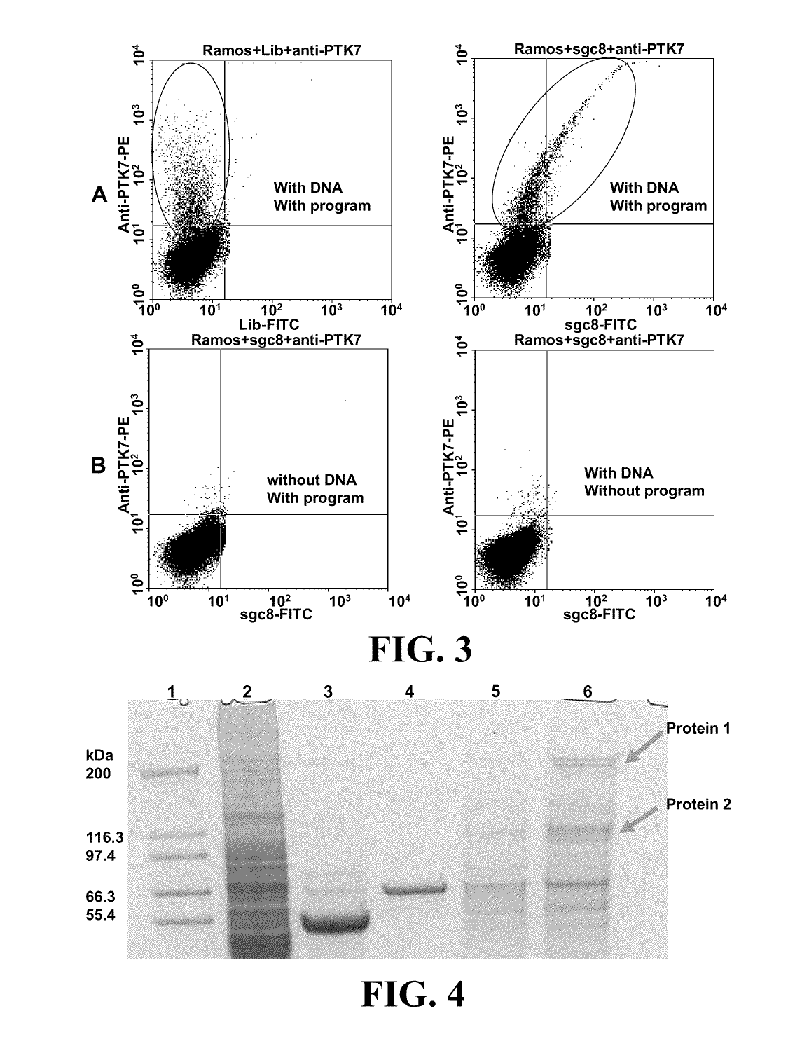

Additional experiments were performed to confirm the target of sgc8. The Ramos cells, which did not show any binding to sgc8 as shown in FIG. 3, were transfected with cDNA of PTK7-1. The transfected Ramos cells were tested with FITC-labeled sgc8 and anti-PTK7-PE by flow cytometry (FIG. 3). About 10% of the Ramos cells were found to express PTK7 after transfection. The transfection also resulted in sgc8 binding of the cells and a very similar linear relationship between FITC and PE signals as previously seen on CEM cells. Another cell line, Toledo cells, which had no interaction with sgc8, was subject to the same transfection procedures, and gave similar results as for Ramos cells.

Previous results with clinical patient samples revealed considerable levels of specific sgc8 binding to leukemia cells closely related to CCRF-CEM cells. Other reports also demonstrated that PTK7 was highly expressed on acute myeloid leukemia samples (C. Muller-Tidow et al., Clin. Cancer Res. 10, 1241 (2004)). Therefore, a link between PTK7 over-expression and leukemia is clearly implied. Discovery of a commonly up-regulated molecular marker on these cells and the development of an excellent aptamer probe for it may lead to effective and reliable diagnosis, as well as cell-specific delivery of therapeutic agents. Previously, a biomarker would be considered ideal if it has high specificity, which means only the patients with the specific tumor will show the presence of the marker; and high sensitivity, which implies that most patients with such tumor should have this marker. However, it has been increasingly recognized that heterogeneity exists among individual patients, and even patients with the same type of cancer can have very different responses to cancer tests and therapies. This calls for personalized biomarkers to match the right patients for reliable cancer diagnosis and guided treatments.

The strategy of the present invention provides a simple way to achieve discovery of personalized biomarkers that are specific for cells from one patient, thus facilitating the on-going efforts toward personalized medicine. Instead of trying to elucidate all possible molecular fingerprints of whole cells, the cell-SELEX based approach focuses on finding highly expressed molecular differences on cell membranes, which could result in a significantly improved efficiency for the identification of clinically practical biomarkers. In addition, the integration of probe selection and biomarker discovery reduces the time gap between laboratory results and clinical application.

### Probe Development

The most significant advantage of the present invention's methods for producing highly specific and/or selective probes is that the resultant probes recognize subtle molecular level differences among targets in their native state without prior knowledge about disease biomarkers. In certain embodiments, aptamer probes are generated using a modified cell-based SELEX selection process (referred to herein as the cell-SELEX process). Instead of using single molecular target, the cell-SELEX process uses whole cells as targets to select aptamers that can distinguish target cells from control cells (see FIG. 6). A counter-selection strategy is employed to isolate DNA sequences that only interact with the target cells but not the control cells. Through this process, a group of cell-specific aptamers can be selected in a relatively short period (4-6 weeks) without knowing which target molecules are present on the cell surface and without knowing which target molecules might play the most important role in cancer development.

Prior to the subject invention, there was no easy way to produce molecular probes in such a short time for an unknown target. In most diseases, the differences at the molecular level are not readily apparent or the problem is just too complicated to use one or a few biomarkers for the identification of a disease. The subject cell-SELEX process for producing personalized molecular probes generates molecular probes that are able to recognize the native states of multiple biomarkers, generating a molecular level understanding of diseases.

In accordance with the methods described herein, many aptamers with high affinity and specificity for the molecular features on the membrane of the cancer cells were obtained, six of them are listed in Table 1. Their equilibrium dissociation constants are in the nM to sub-nM range. Among the 6 aptamers, sgd5 was selected from Toledo cells, a human diffuse large cell lymphoma cell line (B-cell), while the others were from CCRF-CEM cells, a human acute lymphoblastic leukemia cell line (T-cell).

TABLE 1
Molecular aptamers selected from cell-SELEX for leukemia cells.

| Aptamer Sequence name | Kd (nM) |
|---|---|
| sgc3 | 1.97 |
| sgc4 | 26.6 |
| sgd3 | 3.58 |
| sgc8 | 0.80 |
| sgd2 | 7.2 |
| sgd5 | 70.8 |

(Aptamer sgd5 was selected from Toledo cells, a human diffuse large cell lymphoma cell line (B-cell), and all the other aptamers were from CCRF-CEM cells, a human acute lymphoblastic leukemia cell line (T-cell).

The six selected aptamers were first FITC conjugated for recognition of hematologic cancers and human bone marrow. The binding assays with the cells were conducted in a flow cytometer. Four T-cell leukemia cell lines and five B-cell lymphoma, leukemia or myeloma cell lines, and normal human bone marrow aspirates were chosen for this study. Subpopulations of bone marrow cells were identified in the flow cytometer by their side scatter properties and the expression levels of CD45, CD7, CD10, CD19 and CD45 reported by corresponding antibodies. The following cell types were identified: mature B cells, immature B-cells, CD3(+) T-cells, lymphocytes, monocytes, granulocytes, nucleated erythrocytes and early precursors (blast region).

As shown in Table 2 below, aptamer sgd5 only recognized its target cells and a few B-cell lines. All of the cultured T-cell leukemia cell lines were identified by all aptamers except sgd5 with relatively high fluorescence intensity, which was expected as they were selected from a T-cell leukemia cell line. Aptamers sgc8, sgc3, sgd3, sgd5 had almost no binding to either the normal hematopoietic cells in the human bone marrow samples or most B-cells, showing good selectivity toward T-cells. Further inspection showed that aptamers sgc4 and sgd2 were able to recognize many different cell samples including most B-cell lines and some bone marrow cells, indicating the presence of common binding entities on these cells. Combination of the selected aptamers has constructed distinct patterns for different tumor cells, revealing the potential of using aptamers to define molecular signatures of tumors.

TABLE 2
Aptamers binding to cultured cells.

| | | sgc8 | sgc3 | sgc4 | sgd2 | sgd3 | sgd5 |
|---|---|---|---|---|---|---|---|
| T-cell | CCRF-CEM, Pre T ALL | +++ | ++ | ++++ | ++++ | ++ | 0 |
| leukemia | Molt-4, pre T ALL | ++++ | +++ | ++++ | ++++ | ++++ | 0 |
| | Sup-T1, Pre-T ALL. | ++++ | + | ++++ | ++++ | ++ | 0 |
| | Jurkat, Pre-T ALL | ++++ | +++ | ++++ | ++++ | ++++ | 0 |
| B-cell | SUP-B15, pre-B ALL, Ph+ | + | 0 | ++ | + | 0 | 0 |
| leukemia | | | | | | | |
| B-cell | U266, plasmacytoma | 0 | 0 | 0 | 0 | 0 | 0 |
| myeloma | | | | | | | |
| B-cell | Ramos, Burkitt lymphoma | 0 | 0 | ++++ | ++++ | 0 | 0 |
| lymphoma | Toledo, Diffuse large B cell lymphoma | 0 | 0 | ++++ | ++++ | + | ++ |
| | Follicular large B cell lymphoma | 0 | 0 | + | 0 | 0 | 0 |
| | Mo2058, Mantle cell lymphoma | 0 | ++ | ++ | 0 | + | 0 |
| AML | NB-4 (APL) | 0 | 0 | +++ | ++++ | 0 | 0 |
| | Kasumi-1 | ++ | 0 | ++++ | ++++ | + | 0 |
| Bone | CD3 (+) T cells | 0 | 0 | 0 | 0 | 0 | 0 |
| marrow | mature B cellsₐ | 0 | 0 | 0 | 0 | 0 | 0 |
| | Immature B cells_{b} | 0 | 0 | + | + | 0 | 0 |
| | Granulocytes | 0 | 0 | 0 | 0 | 0 | 0 |
| | Monocytes | 0 | 0 | + | + | 0 | 0 |
| | Erythrocytes | 0 | 0 | ++ | ++ | 0 | 0 |
| | Blast | 0 | 0 | + | + | 0 | 0 |

Key:
++++: >85%
+++: 60-85%
++: 35-60%
+: 10-35%
0: <10%
AML: acute myeloid leukemia; ALL: acute lymphoblastic leukemia.

In the flow cytometry analysis, a threshold based on fluorescence intensity of FITC was chosen so that 99 percent of cells incubated with the FITC-labeled unselected DNA library would have fluorescence intensity below it. When FITC-labeled aptamer was allowed to interact with the cells, the percentage of the cells with fluorescence above the set threshold was used to evaluate the binding capacity of the aptamer to the cells. 0 for <10%; + for 10-35%; ++ for 35-60%; +++ for 60-85%; ++++ for >85%.

After showing excellent cellular recognition capabilities with cells, these aptamers were then tested in real leukemia patient samples grouped into different categories, T-ALL, B-ALL, AML, and other lymphomas of mature lymphocytes based on surface markers recognized by antibodies. The results, shown in Table 3 below, clearly demonstrate an effective detection of targets on the cell membranes in patient samples by the selected aptamers. This recognition was not due to non-specific interactions or random binding. All the lymphoma cases showed no or very low binding, in agreement with the fact that the mature lymphoma cells often do not share the same receptors with the immature leukemia cells. Moreover, the aptamers obviously had much stronger binding with the T-ALL cases than others did as expected since the aptamers were selected for the CCRF-CEM cells, a T-ALL cell line. Aptamer binding patterns corresponded well with general categories pre-defined by antibodies.

TABLE 3
Cells from cancer patients.

| | | | | | | |
|---|---|---|---|---|---|---|
| Patient's samples | sgc8 | sgc3 | sgc4 | sgd2 | sgd3 | sgd5 |

T-ALL (T cell acute lymphoblastic leukemia)

| | | | | | | |
|---|---|---|---|---|---|---|
| T ALL 1 | ++ | +++ | +++ | +++ | +++ | ND |
| T ALL 2 | ++ | + | +++ | ++ | + | 0 |
| T ALL 3 | + | + | ++++ | +++ | + | 0 |
| T ALL 4 | + | + | ++ | +++ | + | 0 |
| T ALL 5 | + | + | ++ | + | + | 0 |
| T ALL 6 | 0 | 0 | + | + | 0 | 0 |
| T ALL 7 | 0 | 0 | ++ | ++ | 0 | 0 |

B-ALL (B cell acute lymphoblastic leukemia)

| | | | | | | |
|---|---|---|---|---|---|---|
| B ALL 1 | 0 | 0 | ++ | ++ | 0 | 0 |
| B ALL 2 | 0 | 0 | ++ | ++ | 0 | + |
| B ALL 3 | ++ | 0 | ++ | ++ | 0 | + |

AML (acute myeloid leukemia)

| | | | | | | |
|---|---|---|---|---|---|---|
| AML 1 | + | + | ++ | + | 0 | 0 |
| AML 2 | + | 0 | ++ | + | 0 | 0 |
| AML 3 | + | 0 | + | + | 0 | 0 |
| AML 4 | 0 | 0 | ++++ | ++++ | 0 | 0 |
| AML 5 | 0 | 0 | + | 0 | 0 | 0 |
| AML 6 | + | 0 | 0 | 0 | 0 | 0 |

Others clinical samples

| | | | | | | |
|---|---|---|---|---|---|---|
| 1, T-cell lymphoma | 0 | 0 | 0 | ND | ND | ND |
| 2, follicular | 0 | 0 | 0 | 0 | 0 | 0 |
| lymphoma | | | | | | |
| 3, B-cell lymphoma | 0 | 0 | 0 | 0 | 0 | 0 |
| 4, T-cell | 0 | 0 | 0 | 0 | 0 | 0 |
| lymphoma, | | | | | | |
| 5, B cell lymphoma | 0 | 0 | 0 | 0 | 0 | 0 |
| 6, plasma cell | 0 | 0 | + | + | 0 | 0 |
| neoplasm | | | | | | |

In the flow cytometry analysis, a threshold based on fluorescence intensity of FITC was chosen so that 99 percent of cells incubated with the FITC-labeled unselected DNA library would have fluorescence intensity below it. When FITC-labeled aptamer was allowed to interact with the cells, the percentage of the cells with fluorescence above the set threshold was used to evaluate the binding capacity of the aptamer to the cells. 0 for <10%; + for 10-35%; ++ for 35-60%; +++ for 60-85%; ++++ for >85%.

Despite the results in Table 3 showing little similarity in aptamer binding patterns between cell groups, cases in the same category pre-determined by antibodies could also have quite different patterns. While there is no clear explanation for such differences presently, they precisely reflect the complex nature of the disease. In addition to general categorization of the leukemia defined by antibodies, the aptamer analyses were able to provide extra and valuable information as the direct evidence for the subtle molecular differences among the same type of cancers. It is well known that diseases of the same category could have very different responses to specific treatments (Alizadeh A. A. et al. Distinct types of diffuse large B-cell lymphoma identified by gene expression profiling. Nature 403, 503-511 (2000), Espina, V., Geho, D., Mehta, A. I., Petricoin, E. F. 3rd, Liotta, L. A., & Rosenblatt, K. P. (2005) Cancer Invest. 23, 36-46), but rarely confirmation at the molecular level was offered to classify such dissimilarities. This is partially due to the difficulties in generating effective probes for molecular signatures of diseases using current technologies. In contrast, the cell-SELEX process of the subject invention produces a panel of aptamers quickly and at low cost even when there is no information about disease biomarkers. The resulting aptamers can be used successfully in differentiating closely related cancers. The production of a similar panel of antibodies is almost impossible.

Aptamers directly evolved from tumor cells in accordance with the subject invention can effectively detect various diseases, including leukemia, in patient samples, and even recognize the subtle molecular level differences among (leukemia) patients. The results of experiments conducted to that effect, as seen in Example 2, offer clear evidence that cell-based aptamer selection can be a valuable approach for generating aptamer probes to obtain molecular signature of individual patient samples, forming the foundation for personalized medicine and leading to early diagnosis and highly effective therapy with minimum side effects. Combined with features of aptamers such as chemical-synthesis-based production, low molecular weight, easy modification and long-term stability, the cell-SELEX process of the present invention provides a promising solution for many challenges facing modern medicine. The selected aptamers can also be employed to isolate the disease-specific protein targets to facilitate discovery of clinically important cellular biomarkers.

The following example illustrates a procedure for practicing the invention. This example should not be construed as limiting the scope of the invention in any way. All percentages are by weight and all solvent mixture proportions are by volume unless otherwise noted.

### EXAMPLE 2

### Cell-SELEX:

Cell-based SELEX procedure (Aptamer selection): The cell-SELEX procedure is schematically shown in FIG. 6. Synthesized ssDNA library was incubated with target cells. After washing, the bound DNAs were eluted by heating. The collected DNAs were then incubated with negative cells for counter-selection in order to remove the sequences binding to coexisting molecules on both cells. After centrifugation, the supernatant was collected and the selected DNA pool was amplified by PCR. The PCR products were separated into ssDNAs for next round selection. After the DNA pool reached certain cell-binding affinity, the enriched pool was cloned and sequenced. Aptamers were identified from the sequenced pool. These aptamers have Kd in the range of nM to pM: sgc3: 1.97 nM, sgc4: 26.6 nM, sgc8: 0.8 nM, sgd2: 7.2 nM, sgd3: 3.58 nm, and sgd5: 70.8 nM. Aptamer sgd5 was selected from Toledo cells, a human diffuse large B cell lymphoma cell line. All the other aptamers were from CCRF-CEM cells, a human precursor T cell acute lymphoblastic leukemia cell line.

### Cell Lines.

CCRF-CEM (CCL-119, T-cell lines, human acute lymphoblastic leukemia), Ramos, (CRL-1596. B-cell line, human Burkitt's lymphoma), Toledo (CRL-2631, B-cell line, human diffuse large cell lymphoma), Jurkat (TIB-152, human acute T cell leukemia), Molt-4 (CRL-1582, T-cell lines, human acute lymphoblastic leukemia), Sup-T1 (CRL-1942, T-cell lines, human lymphoblastic leukemia), U266 (TIB-196, B-lymphocyte, human myeloma, plasmacytoma), SUP-B15 (CRL-1929, B-lymphoblast, human acute lymphoblastic leukemia) were obtained from ATCC. All the cell lines were cultured in RPMI 1640 medium (ATCC) supplemented with 10% fetal bovine serum (BSA) (heat inactivated, GIBCO) and 100 IU/mL penicillin-Streptomycin (Cellgro).

### Flow Cytometry Profiling of Leukemia Cells.

FITC labeled aptamers were mixed with PE or PerCP labeled antibodies of CD2, CD3, CD4, CD5, CD7, CD10, CD19, and CD45 respectively, and incubated with 5×10⁵ CCRF-CEM cells and/or 5×10⁵ cells in human bone marrow aspirates in 200 μL binding buffer (Dulbecco's phosphate buffered saline with calcium chloride, magnesium chloride 4.5 g/L glucose, yeast tRNA (0.1 mg/mL) and BSA (1 mg/mL)) for 50 min. Cells were washed twice with 0.7 ml of binding buffer (with 0.1% NaN2), and suspended in 0.4 ml of binding buffer (with 0.1% NaN2). The fluorescence was determined with a FACScan cytometer (Becton Dickinson Immunocytometry systems, San Jose, Calif.) by counting 30000 or 100000 events. The FITC-labeled unselected ssDNA library was used as negative control. A threshold gate based on fluorescence intensity of FITC was set such that 99 percent of negative control cells are below it. The results were expressed as the percentage of cells stained by aptamers with FITC fluorescence above the threshold. Table 2 shows the results of using the aptamers to profile different leukemia cell lines.

### Flow Cytometry Profiling of Patient Samples.

Fresh human lymphoid tissues, bone marrow and peripheral blood samples were from the Hematopathology service, the Department of pathology, University of Florida. Tissue cell suspensions were prepared by mincing the tissue with scalpels in RPMI media, and filtering through a wire mesh screen (#80 mesh). Erythrocytes in the peripheral blood and bone marrow cell suspensions were lysed with ammonium chloride lysing solution (BD Biosciences) for 10 min at room temperature at a volume ratio of 1:9 (sample:lysing) solution. After incubation, cells were pelleted by centri fugation (500 g for 5 min at room temperature). The cells were washed twice in a PBS solution containing 0.1% NaN3, and then resuspended in RPMI medium with 10% FCS and antibiotics. These cells were incubated with aptamers and analyzed by flow cytometry as described above.

### Cell-SELEX for Enrichment of Apatmer Candidates for Target Cells.

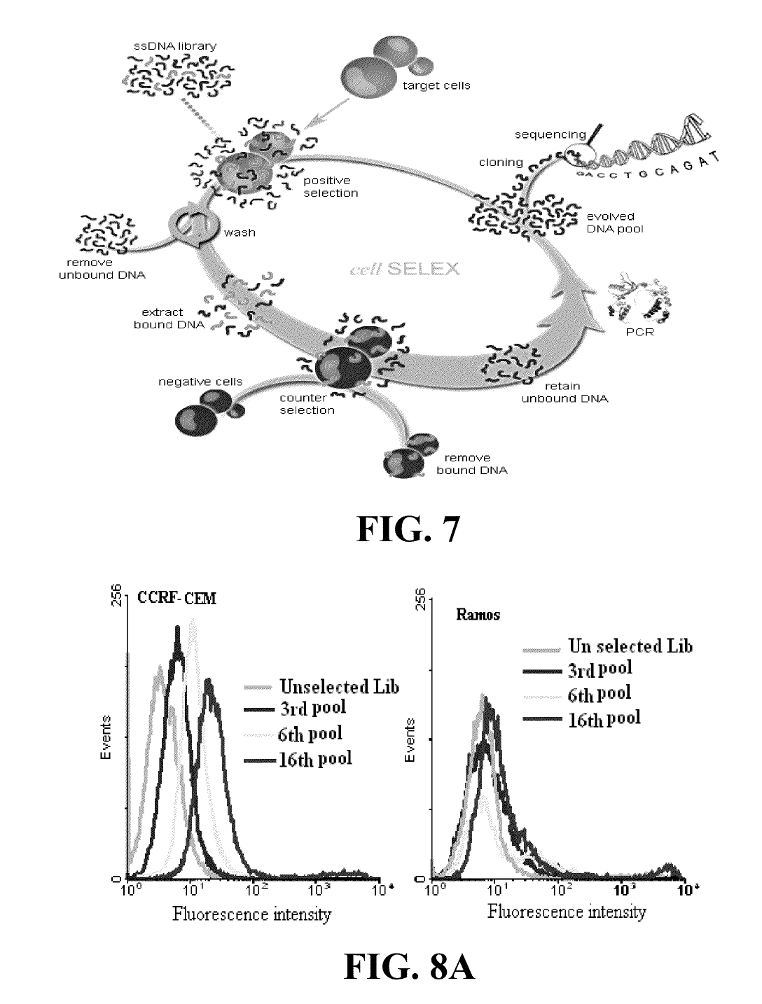

The process of the subject cell-SELEX is illustrated in FIG. 7, and the detailed procedures are provided in the experimental section. Two hematopoietic tumor cell lines were chosen as a model system for aptamer selection because they are well studied and consist of relatively homogeneous tumor cells. In addition, flow cytometry analysis can be easily carried out to monitor the selection process and to evaluate the selected aptamers for their capability of recognizing target cells. Cultured precursor T cell acute lymphoblastic leukemia (ALL) cell line, CCRF-CEM, was used as targets for aptamer selection. A B-cell line from human Burkitt's lymphoma, Ramos, was used as the negative control to reduce collection of DNA sequences that could bind to common surface molecules present on both types of cells.

During selection, a library of single-stranded DNAs that contained a 52-mer random sequence region flanked by two 18-mer PCR primer sequences was used. The library was incubated with the target cells to allow binding to take place. Then the cells were washed and the DNA sequences bound to the cell surface were eluted. The collected sequences were then allowed to interact with excess negative control cells and only the DNA sequences remained free in the supernatant were collected and amplified for the next round selection. After multi-round selection, the subtraction process efficiently reduced the DNA sequences that bound to the control cells, while those target-cell-specific aptamer candidates were enriched.

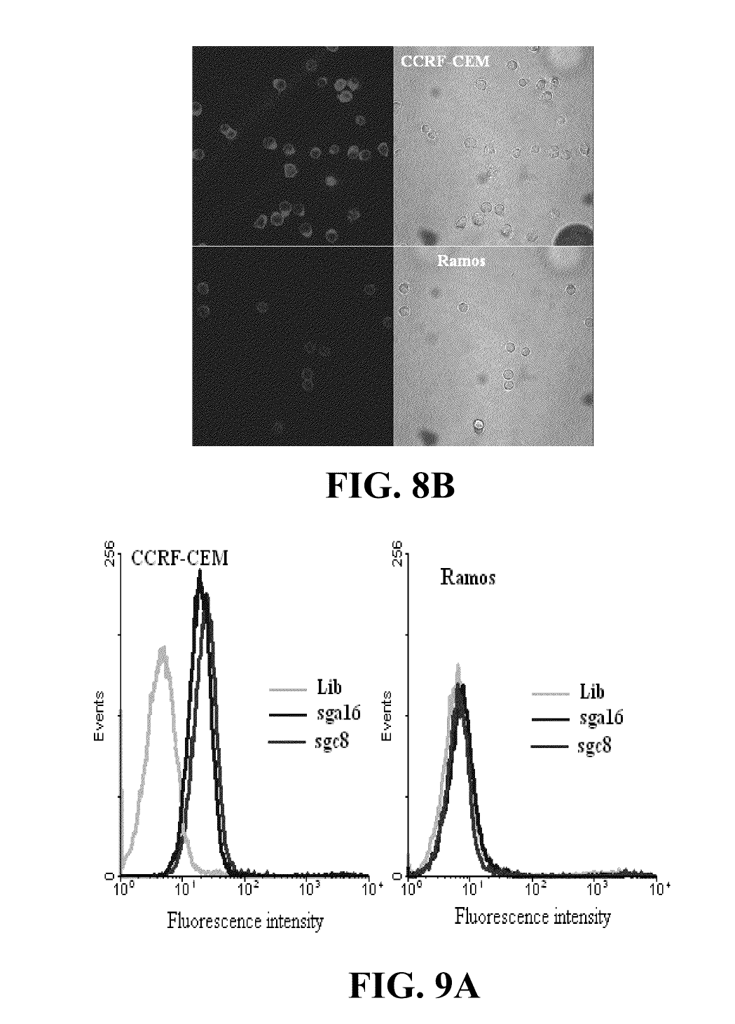

The progress of the selection process was monitored using flow cytometry. DNA products collected after each round were labeled with fluorescein isothiocyanate (FITC) dye and incubated with live cells. The fluorescence intensity of the labeled cells measured by the flow cytometry analysis represented binding capacity of the enriched DNA pool to the cells. With increasing number of selection cycles, steady increases in fluorescence intensity on the CCRF-CEM cells (target cells) were observed (FIG. 8A), indicating that DNA sequences with better binding affinity to the target cells were enriched. Nevertheless, there was no significant change in fluorescence intensity on the Ramos cells (control cells). These results indicate that the DNA probes specifically recognizing unique surface targets on CCRF-CEM cells were isolated. The specific binding of the selected pools to the target cells was further confirmed by confocal imaging (FIG. 8B). After incubation with tetramethylrhodamine (TAMRA) dye-labeled aptamer pool, the CEM cells presented very bright fluorescence on the periphery of cells, while the Ramos cells displayed weak fluorescence.

### Identification of Aptamers for the Target Cells.

Usually it took about 20 rounds to achieve excellent enrichment of aptamer candidates. The highly enriched aptamer pools were cloned and sequenced by a high-throughput Genome Sequencing method. The sequences were grouped based on the homology of the DNA sequences of individual clones with each group containing very similar sequences.

Twenty sequences were chosen for further characterization because they were highly abundant in their family. The binding assays of the selected sequences with target cells were performed using flow cytometry. Thirteen sequences revealed obvious binding to CCRF-CEM cells. Moreover, the binding was not interfered by the addition of 1000 folds of starting DNA library. Except aptamers such as sgd2, sgc4 and its homologue sgc4a, which could recognize both CCRF-CEM and Ramos cells, other aptamers only recognized the target cell line, CCRF-CEM. Ten aptamers were confirmed to have high affinity for CCRF-CEM cells with calculated equilibrium dissociation constants (Kds) in the nM to pM range and their Kds are listed in Table 4 below. CD2, CD3, CD4, CD5, CD7, CD45 are the surface antigens expressed on CCRF-CEM cells, and none of the tested aptamer sequences showed any evidence of competition with antibodies against these antigens. This indicates that the aptamers may interact with unique surface binding entities.

TABLE 4
Sequences and Kds of the selected aptamers:
5′-ATA CCA GCT TAT TCA ATT-(center random
sequence)-AGA TAG TAA GTG CAA TCT-3′

| Sequence | Center | Kd |
|---|---|---|
| name | sequence* | (nM) |
| sgc3 | CCTGTGGGAAGGCTATAGAGGGGCCAGT | 1.97 ± 0.29 |
| | CTATGAATAAGATGGCGGACTAATGTGT | |
| | A (SEQ ID NO: 6) | |
| sgc6 | CCTGTGGGAAGGCTATAGAGGGGCCA | 8.76 ± 0.62 |
| | GTCTATGAACAAGATGGTTGATCCGT | |
| | (SEQ ID NO: 7) | |
| sgd3 | AGGGGGAGCTTGCGCGCATCAAGGTG | 3.58 ± 0.58 |
| | CTAAACGAAAGCCTCATGGCTTCTAT | |
| | (SEQ ID NO: 8) | |
| sgc4 | CGAGTGCGGATGCAAACGCCAGACAGG | 26.6 ± 2.1 |
| | GGGACAGGAGATAAGAATAGCGTGATG | |
| | (SEQ ID NO: 9) | |
| sgc4a | CGAGTGCGGATGCAAACG | 229 ± 38 |
| | CCAGACAGGGGGACAGG | |
| | (SEQ ID NO: 10) | |
| sgc5 | ACCGACGACGAACTATCTATCA | 113 ± 41 |
| | CTATCTTACACATCATACCTCG | |
| | (SEQ ID NO: 11) | |
| sgc7 | ACCGCAGCGACTATCTCGACTACATT | 144 ± 75 |
| | ACTAGCTTATACTCCGATCATCTCT | |
| | (SEQ ID NO: 12) | |
| sgc8 | AGTCACACTTAGAGTTCTAACTGCTG | 0.80 ± 0.09 |
| | CGCCGCCGGGAAAATACTGTACGGTT | |
| | (SEQ ID NO: 13 | |
| sga16 | AGTCACACTTAGAGTTCTAGCTGCTG | 5.00 ± 0.52 |
| | CGCCGCCGGGAAAATACTGTACGGAT | |
| | (SEQ ID NO: 14) | |
| Sgd2 | GAGTGAAGCAAGGATGCAACCTCGGC | 7.21 ± 0.89 |
| | TCCAACCCGTGAGAGTCGCGAAACTC | |
| | (SEQ ID NO: 15) | |

The full-length sequences include two primer hybridization sites and the center random sequence.

### Cell-SELEX Generates High Affinity Molecular Probes for Target Cells.

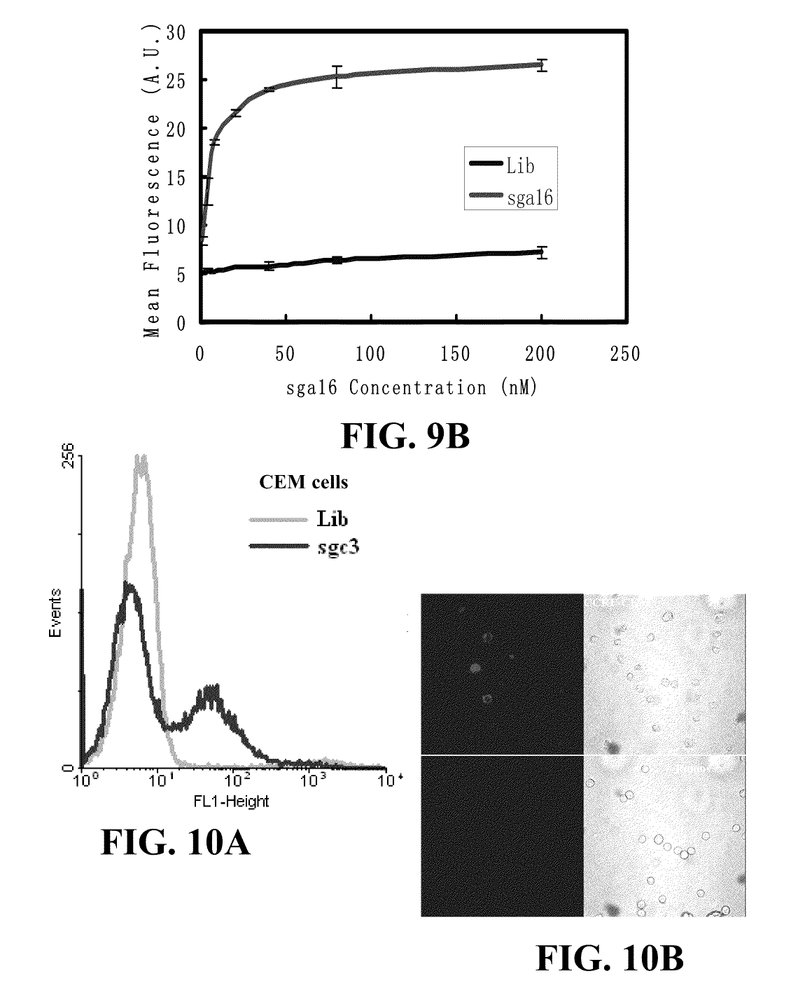

The individual aptamers were tested. As shown in FIGS. 9A and 9B, aptamers sga16 (Kd=5.00±0.52 nM) and its homologues sgc8 (Kd=0.80±0.09 nM) can specifically recognize the CCRF-CEM cells with high affinity. The specificity of both aptamers was also observed directly using confocal imaging. Intense fluorescence from sga16 bound on the CCRF-CEM cell surface was observed, while the Ramos cells had no obvious fluorescence. These results have clearly demonstrated the great potential of using aptamer sga16 and sgc8 as excellent molecular probes for CCRF-CEM cell recognition.

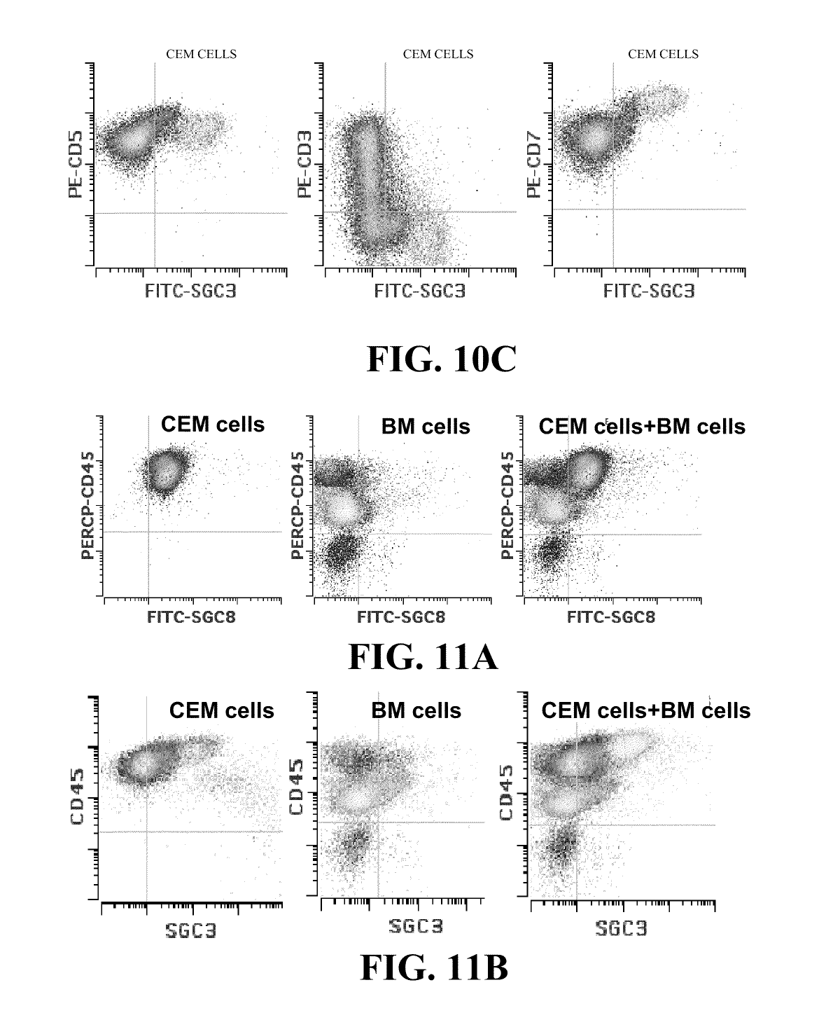

It is worth noting that some of the selected aptamers can identify binding entities expressed only by a small subset of target cells. Sequences sgc3 (Kd=1.97±0.29 nM), sgc6 (Kd=8.76±0.62 nM) and sgd3 (Kd=3.58±0.58 nM) were found to bind only to a small population of the CCRF-CEM cells (about 20%-40% of the cells) (the second peak in FIG. 10A and yellow area in FIG. 10C) with high affinity, but they did not bind to Ramos cells. Sgc3 and sgc6 are in fact homologues, while sequence sgd3 is very different from them. Confocal imaging also confirmed that aptamer sgc3 strongly bound to a subset of CCRF-CEM cells (FIG. 10B). These sgc3-labeled cells showed the same forward scatter and side scatter properties as the rest of sgc3-negative cells in flow cytometry assays, indicating they were viable cells. The sgc3-labeled cells were immunophenotyped and it was confirmed that these cells were CD5-positve and CD7-positive neoplastic T cells rather than other cell contamination (FIG. 10C). On the other hand, the sgc3-binding CCRF-CEM cells were CD3-negative, implying that they might represent a unique differentiation stage or phase of cell cycles.

### The Selected Aptamers Can be Used for Highly Specific Recognition of Target Cells in Real Biological Samples.

To test the feasibility of the selected aptamers as probes for specific molecular recognition, FITC-labeled aptamers (sgc8, sgc3, sgd3, sgc4, sgd2) and monoclonal antibodies were used to detect CCRF-CEM leukemia cells mixed with normal human bone marrow aspirates. The human bone marrow aspirates consisted of mature and immature granulocytes, nucleated erythrocytes, monocytes, mature and immature B cells, and T cells. As expected, sgc8, sgc3 and sgd3 only recognized cultured leukemia T cells (CCRF-CEM) (FIG. 11), and did not bind to normal CD3-positive T cells or any other bone marrow cells. Aptamers sgc4 and sgd2 slightly hound to mature and immature B cells, a subset of CD3-positive T cells, nucleated erythrocytes from the human bone marrow, and cultured leukemia T cells (CCRF-CEM) (data not shown).

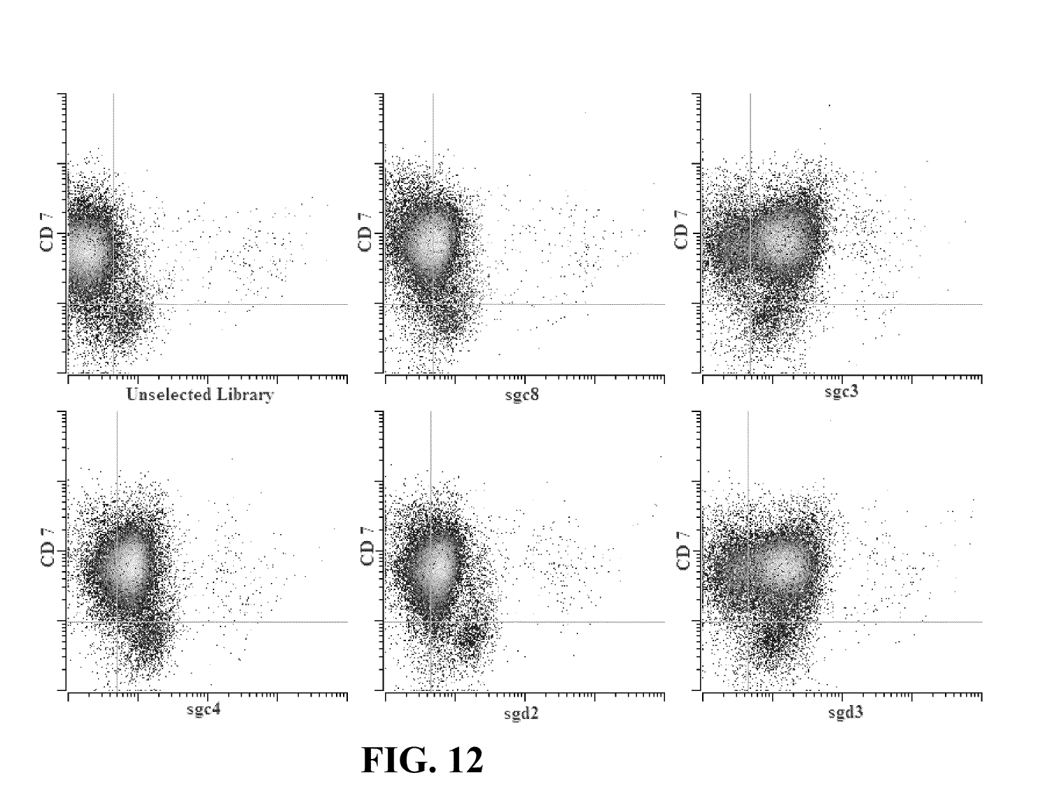

Aptamers (sgc8, sgc3, sgd3, sgc4, sgd2) were all found to recognize other T-cell acute lymphoblastic leukemia (ALL) cell lines, Sup-T1, Molt-4, and Jurkat, but not all of them could bind to cultured B-cells and AML cells (Table 5 below). Recognition of tumor cells in real clinical samples by the these aptamers was also tested. Patient's bone marrow aspirates were examined with FITC-labeled aptamers and monoclonal antibodies. The results (Table 5) revealed that none of aptamers (sgc8, sgc3, sgd3, sgc4, sgd2) could recognize the cancer cells from B-cell lymphoma patient, but all of them were able to bind the cancer cells from T cell ALL patient (FIG. 12), which were closely related to the CCRF-CEM target cells used in the subject cell-SELEX process. It can be seen that aptamers that specifically bind to the CCRF-CEM target cells could also recognize cells closely related to the target cell line even in real clinical samples. The capability of the aptamers selected in the subject cell-SELEX process for molecular diagnosis in clinical practice is clearly demonstrated here.

TABLE 5
Using aptamers to recognize cancer cells

| | Cell line | sgc8 | sgc3 | sgc4 | sgd2 | sgd3 |
|---|---|---|---|---|---|---|
| Cultured | Molt-4 (T cell-ALL) | ++++ | +++ | ++++ | ++++ | ++++ |
| cell lines | Sup-T1 (T cell-ALL) | ++++ | + | ++++ | ++++ | ++ |
| | Jurkat (T cell-ALL) | ++++ | +++ | ++++ | ++++ | ++++ |
| | SUP-B15 (B cell-All) | + | 0 | ++ | + | 0 |
| | U266 (B-cell myeloma) | 0 | 0 | 0 | 0 | 0 |
| | Toledo (B-cell | 0 | 0 | ++++ | ++++ | + |
| | lymphoma) | | | | | |
| | Mo2058 (B-cell | 0 | ++ | ++ | 0 | + |
| | lymphoma) | | | | | |
| | NB-4 (AML, APL) | 0 | 0 | +++ | ++++ | 0 |
| Cells from | TALL | ++ | +++ | +++ | +++ | +++ |
| patients | Large B-cell lymphoma | 0 | 0 | 0 | 0 | 0 |

Note.
A threshold based on fluorescence intensity of FITC in the flow cytometry analysis was chosen so that 99 percent of cells incubated with the FITC-labeled unselected DNA library would have fluorescence intensity below it. When FITC-labeled aptamer was allowed to interact with the cells, the percentage of the cells with fluorescence above the set threshold was used to evaluate the binding capacity of the aptamer to the cells. 0: <10% +: 10-35%, ++: 35-60%, +++: 60-85%, ++++: >85%; AML: acute myeloid leukemia; ALL: acute lymphoblastic leukemia, APL: acute promyelocytic leukemia

### The Binding Sites of the Aptamers on the Target Cells are Most Likely Proteins.

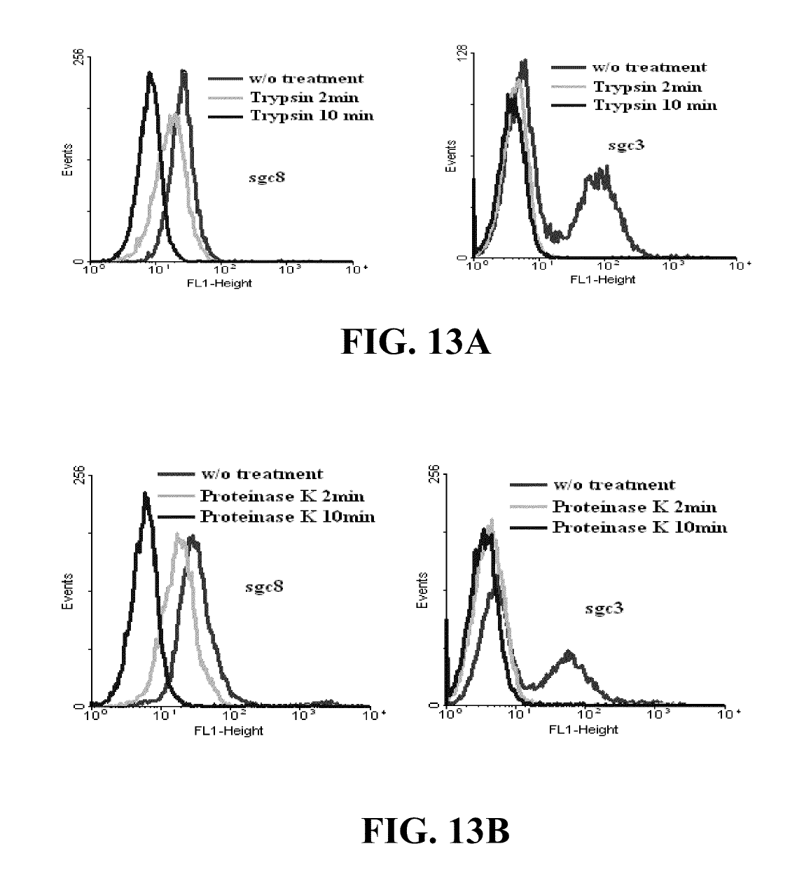

To have a preliminary test whether the targets of the aptamers are membrane proteins on the cell surface, CCRF-CEM cells were treated with proteinases such as trypsin and proteinase K for a short time before adding the aptamer to these treated cells. As shown in FIG. 13, after treating the cells with trypsin or proteinase K for 10 minutes, aptamer sgc8, sgc3, and sgd3 lost their binding to these cells, while the interactions of aptamer sgd2 and sgd4 with the cells were not affected. It can be deduced that the binding entities of aptamer sgc8, sgc3 and sgd3 had been removed by the proteinases, indicating the target molecules were most likely membrane proteins. Interestingly, the targets of aptamers sgd2 and sgc4 were clearly not affected by the proteinases.

According to the subject invention, a cell-based SELEX strategy is provided to generate a panel of aptamers as useful molecular probes to reveal the molecular level differences between any two types of cells. The selected aptamers have then been used for the specific recognition of diseased cells. Molecular differences between the target and control cells could be easily isolated, providing an effective approach to the discovery of molecular signatures of many other diseases. More importantly, detailed knowledge of the distinct targets on the cell surface is not needed prior to the selection, which could greatly simplify the process of molecular probe development.

The entire selection process of the subject invention is simple, fast, reproducible and straightforward, and the selected aptamers can specifically bind to target cells with Kds in the nM to pM range. Some of the aptamers can recognize a small subset of the target cells. Target cells mixed with normal human bone marrow aspirate can be readily distinguished. In addition, cancer cells from clinical patients' specimens, which are closely related to the target cells were also recognized by the selected aptamers. Furthermore, the aptamers can be employed to isolate the disease-specific protein targets to facilitate discovery of clinically important biomarkers.

By developing specific probes for molecular signatures on cancer cell surface in accordance with the subject invention, the user is afforded the ability to define tumors, create tailored treatment regime for more “personalized” medicine, monitor the response to therapy, and detect minimal residual diseases.

The following example illustrates a procedure for practicing the invention. This example should not be construed as limiting the scope of the invention in any way. All percentages are by weight and all solvent mixture proportions are by volume unless otherwise noted.

### EXAMPLE 3

### Cell Lines and Buffers

CCRF-CEM (CCL-119, T-cell lines, human acute lymphoblastic leukemia), Ramos, (CRL-1596, B-cell line, human Burkitt's lymphoma), Toledo (CRL-2631, B-cell line, human diffuse large cell lymphoma), Sup-T1(CRL-1942, T-cell lines, human lymphoblastic leukemia), Jurkat (TIB-152, human acute T cell leukemia), Molt-4 (CRL-1582, T-cell lines, human acute lymphoblastic leukemia), SUP-B15 (CRL-1929, B-lymphoblast, human acute lymphoblastic leukemia) and U266 (TIB-196, B-lymphocyte, human myeloma, plasmacytoma) were obtained from ATCC (American Type Culture collection). Mo2058 (Mantle-cell lymphoma, Epstein-Barr Virus-positive cell line) and NB-4 (acute promyelocytic leukemia) were obtained from Department of Pathology, University of Florida). All the cells were cultured in RPMI 1640 medium (ATCC) supplemented with 10% fetal bovine serum (FBS) (heat inactivated, GIBCO) and 100 IU/mL penicillin-Streptomycin (Cellgro). Cells were washed before and after incubation with wash buffer (4.5 g/L glucose and 5 mM MgCl₂ in Dulbecco's phosphate buffered saline with calcium chloride and magnesium chloride (Sigma)). Binding buffer used for selection was prepared by adding yeast tRNA (0.1 mg/mL) (Sigma) and BSA (1 mg/mL) (Fisher) into wash buffer to reduce background binding. Antibodies against CD2, CD3, CD4, CD5, CD7 and CD45 were purchased from BD Biosciences. Trypsin and proteinase K were purchased from Fisher biotech.

### SELEX Library and Primers

HPLC purified library contained a central randomized sequence of 52 nucleotides (nt) flanked by two 18-nt primer hybridization sites (SEQ ID NOS:1-2) (5′-ATA CCA GCT TAT TCA ATT (SEQ ID NO:1)-52-nt-AGA TAG TAA GTG CAA TCT-(SEQ ID NO:2)3′). A fluorescein isothiocyanate (FITC)-labeled 5′-primer (5′-FITC-ATA CCA OCT TAT TCA ATT (SEQ ID NO:16)-3′) or a tetramethylrhodamine anhydride (TAMRA)-labeled 5′-primer (5′-TMR-ATA CCA GCT TAT TCA ATT (SEQ id NO:17)-3′); and a triple biotinylated (trB) 3′-primer (5′-trB-AGA TTG CAC TTA CTA TCT (SEQ ID NO:18)-3′) were used in the PCR reactions for the synthesis of double-labeled, double-stranded. DNA molecules. After denaturing in alkaline condition (0.2 M NaOH), the FITC-conjugated sense ssDNA strand was separated from the biotinylated anti-sense ssDNA strand with streptavidin-coated sepharose beads (Amersham Bioscience) and used for next round selection. The selection process was monitored using flow cytometry.

### SELEX Procedures

The procedures of selection were as follows: ssDNA pool (200 pmol) dissolved in 400 μL binding buffer was denatured by heating at 95° C. for 5 min and cooled on ice for 10 min before binding. Then the ssDNA pool was incubated with 1-2×10⁶ CCRF-CEM cells (target cells) on ice for 1 hour. After washing, the bound DNAs were eluted by heating at 95° C. for 5 min in 300 μL of binding buffer. The eluted DNAs were then incubated with Ramos cells (negative cells, 5-fold excess than CCRF-CEM cells) on ice for counter-selection for 1 hour. After centrifugation, the supernatant was desalted and then amplified by PCR with FITC- or biotin-labeled primers (10-20 cycles of 0.5 min at 94° C., 0.5 min at 46° C., and 0.5 min at 72° C., followed by 5 min at 72° C.; the Taq-polymerase and dNTPs were obtained from Takara). The selected sense ssDNA was separated from the biotinylated anti-sense ssDNA strand by streptavidin-coated sepharose beads (Amersham Bioscience). For the first round selection, the amount of initial ssDNA pool was 10 nmol, dissolved in 1 mL binding buffer; and the counter selection step was eliminated. In order to acquire aptamers with high affinity and specificity, the wash strength was enhanced gradually by extending wash time (from 1 min to 10 min), increasing the volume of wash buffer (from 0.5 mL to 5 mL) and the number of washes (from 3-5). Additionally, 20% FBS and 50-300 fold molar excess genomic DNA were added to the incubation solution. After 20 rounds of selection, selected ssDNA pool was PCR-amplified using unmodified primers and cloned into Escherichia coli using the TA cloning kit (Invitrogen). Cloned sequences were determined by the Genome Sequencing Services Laboratory at the University of Florida.

### Flow Cytometric Analysis

To monitor the enrichment of aptamer candidates after selection, FITC-labeled ssDNA pool was incubated with 2×10⁵ CCRF-CEM cells or Ramos cells in 200 μL of binding buffer containing 20% FBS on ice for 50 min. Cells were washed twice with 0.7 ml of binding buffer (with 0.1% NaN₂), and suspended in 0.4 ml of binding buffer (with 0.1% NaN₂). The fluorescence was determined with a FACScan cytometer (Becton Dickinson Immunocytometry Systems, San Jose, Calif.) by counting 30000 events. The FITC-labeled unselected ssDNA library was used as negative control.

The binding affinity of aptamers was determined by incubating CCRF-CEM cells (5×10⁵) with varying concentrations of FITC-labeled aptamer in 500 μL volume of binding buffer containing 20% FBS on ice for 50 min in the dark. Cells were then washed twice with 0.7 ml of the binding buffer with 0.1% sodium azide, suspended in 0.4 ml of binding buffer with 0.1% sodium azide, and subjected to flow cytometric analysis within 30 min. The FITC-labeled unselected ssDNA library was used as negative control to determine nonspecific binding. All the experiments for binding assay were repeated 2-4 times. The mean fluorescence intensity of target cells labeled by aptamers was used to calculate for specific binding by subtracting the mean fluorescence intensity of non-specific binding from unselected library DNAs (Davis, K. A., Abrams, B., Lin, Y. & Jayasena, S. D. (1996) Nucleic. Acids. Res. 24, 702-706, Davis, K. A., Lin, Y., Abrams, B. & Jayasena, S. D. (1998) Nucleic. Acids. Res. 26, 3915-3924). The equilibrium dissociation constants (Kd) of the aptamer-cell interaction were obtained by fitting the dependence of fluorescence intensity of specific binding on the concentration of the aptamers to the equation Y=BmaxX/(Kd+X) using the SigmaPlot software (Jandel Scientific, San Rafael, Calif.).

To test the feasibility of using aptamers for recognition of cancer cells in real biological samples, FITC labeled aptamers were mixed with PE or PerCP labeled antibodies of CD2, CD3, CD4, CD5, CD7, CD19, and CD45 respectively, and incubated with 2×10⁵ cancer cells and/or 2×10⁵ cells in human bone marrow aspirates. After washing as described above, the fluorescence was determined with a FACScan cytometer (Becton Dickinson Immunocytometry Systems, San Jose, Calif.).

Confocal Imaging of Cell Bound with Aptamer

For confocal imaging, the selected ssDNA pools or aptamers were labeled with TAMRA. Cells were incubated with 50 pmol TMR-labeled ssDNA in 100 μL of binding buffer containing 20% FBS on ice for 50 min. Other treatment steps were the same as described in the flow cytometry selection. 20 μL of cell suspension bound with TAMRA-labeled ssDNA was dropped on a thin glass slide placed above a 60× objective on the confocal microscope and then covered with a cover slip. Imaging of the cells was performed on an Olympus FV500-IX81 confocal microscope (Olympus America Inc., Melville, N.Y.). A 5 mW 543 nM He—Ne laser was the excitation source for TAMRA throughout the experiments. The objective used for imaging was a PLAPO60XO3PH 60× oil immersion objective with a numerical aperture of 1.40 from Olympus (Melville, N.Y.).

### Proteinase Treatment for Cells

5×10⁶ of CCRF-CEM cells were washed with 2 ml PBS, then incubateded with 1 mL of 0.05% Trypsin/0.53 mM EDTA in HBSS or 0.1 mg/ml proteinase K in PBS at 37□ for 2 min and 10 mM. FBS was then added to quench with the proteinases. After washing with 2 ml binding buffer, the treated cells were used for aptamer binding assay as described in the flow cytometric analysis section.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A method for developing a personalized biomarker for a target disease cell comprising:
(a) obtaining a probe having high affinity and selectivity for a biomarker on the membrane of a target cell;
(b) labeling the probe with a biotin tag;
(c) lysing the target cell and extracting membrane content of the target cell;
(d) solubilizing the membrane content;
(e) binding biotin-tagged probe with solubilized membrane content to form a biotin-probe-biomarker complex;
(f) extracting and collecting the biotin-probe-biomarker complex using streptavidin coated magnetic beads and a magnetic stand; and
(g) separating the biotin-magnetic beads and probes from the biomarker.

2. The method of claim 1, further comprising the step of (h) analyzing resultant biomarker to identify the biomarker.

3. The method of claim 2, wherein the analysis step (h) is selected from any one or combination from the group consisting of: analysis by polyacrylamide gel electrophoresis (SDS-PAGE); analysis by HPLC-Mass Spectroscopy; analysis by Liquid Chromatography/Mass Spectrometry/Mass Spectrometry (LC-MS/MS); analysis by comparison of biomarker with those listed in electronic databases; and analysis by flow cytometry.

4. The method of claim 1, wherein the probe is an aptamer.

5. The method of claim 1, wherein the probe is sgc8c.

6. The method of claim 1, wherein the target disease cell is a cancer cell.

7. The method of claim 6, wherein the cancer cell is a leukemia cell.

8. The method of claim 1, wherein the step of solubilizing the membrane content comprises dissolving the membrane content in PBS buffer containing surfactant.

9. A method for developing a personalized biomarker for a disease comprising:
(a) incubating a sample containing at least one nucleic acid sequence with a sample containing at least one target cell;
(b) allowing substantially all of the target cells to bind with the nucleic acid sequences;
(c) separating and recovering bound nucleic acid sequences to form a first sample;
(d) eluting and incubating the first sample with a sample containing at least one counter-selective cell so that the nucleic acid sequences bind with the counter-selective cells;
(f) separating and recovering unbound nucleic acid sequences to form a second sample;
(g) cloning and sequencing the nucleic acid sequences of the second sample to obtain a probe specific for a biomarker on the membrane of the target cell;
(h) labeling the probe with a biotin tag;
(i) lysing the target cell and extracting membrane content of the target cell;
(j) solubilizing the membrane content;
(k) binding biotin-tagged probe with solubilized membrane content to form a biotin-probe-biomarker complex;
(l) extracting and collecting the biotin-probe-biomarker complex using streptavidin coated magnetic beads and a magnetic stand; and
(m) separating the biotin-magnetic beads and probes from the biomarker.

10. The method of claim 9, further comprising the step of (h) analyzing resultant biomarker to identify the biomarker.

11. The method of claim 10, wherein the analysis step (h) is selected from any one or combination from the group consisting of: analysis by polyacrylamide gel electrophoresis (SDS-PAGE); analysis by HPLC-Mass Spectroscopy; analysis by Liquid Chromatography/Mass Spectrometry/Mass Spectrometry (LC-MS/MS); analysis by comparison of biomarker with those listed in electronic databases; and analysis by flow cytometry.

12. The method of claim 9, wherein the step of solubilizing the membrane content comprises dissolving the membrane content in PBS buffer containing surfactant.

13. The method of claim 9, wherein the target disease cell is a cancer cell.

14. The method of claim 13, wherein the cancer cell is a leukemia cell.

15. The method of claim 9, wherein the probe is sgc8c.

16. The method of claim 9, further comprising the steps of:
(f¹) using a quantitative replicative procedure comprising a replicative polymerase reaction following step (f); and
(g¹) repeating steps (a) through (f¹) at least one more time before proceeding to step (g), wherein the greater number of times step (g¹) is performed provides a probe with a higher affinity for the target cell.
